(19) United States
(12) Patent Application Publication
(10) Pub. No.: US 2022/0047869 A1
(43) Pub. Date: Feb. 17, 2022

(54) DEVICE NETWORKS FOR MODULATING NEURAL ACTIVITY

(71) Applicant: Iota Biosciences, Inc., Berkeley, CA (US)

(72) Inventors: Jose M. CARMENA, Alameda, CA (US); Michel M. MAHARBIZ, El Cerrito, CA (US); Ryan NEELY, El Cerrito, CA (US)

(73) Assignee: Iota Biosciences, Inc., Berkeley, CA (US)

(21) Appl. No.: 17/298,116

(22) PCT Filed: Dec. 4, 2019
(86) PCT No.: PCT/US19/64523

§ 371 Date: May 28, 2021

Related U.S. Application Data
(60) Provisional application No. 62/776,351, filed on Dec. 6, 2018.

(51) Int. Cl. A61N 1/36

(52) U.S. Cl. CPC A61N 1/36135; A61N 1/36146

(57) ABSTRACT

Described herein are implantable device networks that include two or implantable devices configured to modulate neural activity in a subject. The network includes at least one implantable device that can detect a detection signal, such as an electrophysiological signal or a physiological condition. The network also includes a second implantable device configured to emit an electrical pulse based at least on information related to the detection signal. The implantable devices in the network can wirelessly communicate between each other, either directly or through an intermediate device.

### CROSS-REFERENCE TO RELATED APPLICATION

This application claims the priority benefit of U.S. Provisional Application No. 62/776,351, filed on Dec. 6, 2018; which is incorporated herein by reference in its entirety for all purposes.

### TECHNICAL FIELD

The present invention relates to an implantable neuromodulation device network, and methods of using such a network.

### BACKGROUND

The peripheral nervous system of an individual operates activity of vital organs and physiological homeostasis with tight control. Electrical pulses transmitted through nerves can alter, for example, heart rates, inflammation, and bladder or bowel control. Certain medical conditions can arise when these neural signals fail to properly control the body, either by over-stimulating or under-stimulating target organs.

Invasive methods have been developed for treating abnormal physiological activity by controlling the electrical signals of the peripheral nervous system. Such methods can include implanting electrodes into the body of a patient, with the tips of the electrodes contacting target nerves. These electrodes generally have long leads that attach to an external device or a bulky implanted device, which subject the patient to substantial risk of infection or displacement of the electrodes. Additionally, because many of the methods are so invasive, certain treatments are limited to clinical settings, and cannot be used as an at-home remedy. Wholly implantable devices have been developed for less invasive treatment, but such devices are too large to be placed in many locations of the body. Therefore, the implanted devices require the use of long leads, which can be displaced or break.

Tightly controlled neuromodulation of targeted nerves for therapeutic purposes relies on a systematic assessment of a subject's physiological condition or physiological signaling. Certain electrophysiological transmissions or physiological conditions (e.g., temperature or analyte concentration) elsewhere in the body can inform whether or how to stimulate a target nerve to obtain a desired therapeutic effect. There continues to be a need for systems that can effectively stimulate the nervous system to obtain therapeutic effects that accounts for other physiological activities within the patient.

The disclosures of all publications, patents, and patent applications referred to herein are each hereby incorporated by reference in their entireties. To the extent that any reference incorporated by reference conflicts with the instant disclosure, the instant disclosure shall control.

### SUMMARY OF THE INVENTION

Described herein are implantable device networks, and methods of modulating neural activity using such implantable device networks.

In some embodiments, a method of modulating neural activity using an implantable device network comprises: (a) detecting, at one or more implantable devices in a first set of one or more implantable devices, a detection signal comprising one or more electrophysiological signals transmitted by a recorded nerve or one or more physiological conditions; (b) wirelessly transmitting, from the one or more implantable devices in the first set of one or more implantable devices, information related to the detection signal; (c) wirelessly receiving, at one or more implantable devices in a second set of one or more implantable devices, the information related to the detection signal; and (d) determining whether to emit, from one or more implantable devices in the second set of one or more implantable devices, one or more electrical pulses configured to modulate neural activity of one or more target nerves based on at least the received information related to the detection signal.

In some embodiments, the method further comprises emitting, at the one or more implantable devices in the second set of one or more implantable devices, the one or more electrical pulses. In some embodiments, the method comprises determining one or more pulse characteristics of the one or more electrical pulses emitted from the one or more implantable devices in the second set of one or more implantable devices in the second set of one or more implantable devices.

In some embodiments, the method comprises wirelessly transmitting, from the one or more implantable devices in the second set of one or more implantable devices, information related to the one or more implantable devices in the second set of one or more implantable devices; wirelessly receiving, at the one or more implantable devices in the first set of one or more implantable devices, the information related to the one or more implantable devices in the second set of one or more implantable devices; and determining whether to emit, from one or more implantable devices in the first set of one or more implantable devices, one or more electrical pulses configured to modulate neural activity of one or more additional target nerves based on at least the information related to the one or more implantable devices in the second set of one or more implantable devices. In some embodiments, the method comprises emitting, at the one or more implantable devices in the first set of one or more implantable devices, the one or more electrical pulses configured to modulate neural activity of the one or more additional target nerves. In some embodiments, determining whether to emit the one or more electrical pulses configured to modulate neural activity of the one or more additional nerves comprises updating a dynamic state of the one or more implantable devices in the first set of one or more implantable devices.

In some embodiments of the method, the information related to the one or more implantable devices in the second set of one or more implantable devices wirelessly transmitted by the one or more implantable devices in the second set of one or more implantable devices comprises information related to a detection signal detected by the one or more implantable devices in the second set of one or more implantable devices. In some embodiments, the information related to the one or more implantable devices in the second set of one or more implantable devices wirelessly transmitted by the one or more implantable devices in the second set of one or more implantable devices comprises information related to a dynamic state of one or more of the implantable devices in the second set of one or more implantable devices. In some embodiments, the information related to the one or more implantable devices in the second set of one or more implantable devices wirelessly transmitted by the one or more implantable devices in the second set of one or more implantable devices comprises information related to the one or more electrical pulses emitted by the one or more implantable devices in the second set of one or more implantable devices.

In some embodiments of the method, determining whether to emit the one or more electrical pulses from the one or more implantable devices in the second set of one or more implantable devices comprises implementing a feedforward neural network process. In some embodiments of the method, determining whether to emit the one or more electrical pulses from the one or more implantable devices in the second set of one or more implantable devices comprises updating a dynamic state of one or more implantable devices in the second set of one or more implantable devices.

In some embodiments of the method, a determination of whether to emit the one or more electrical pulses from the one or more implantable devices in the second set of one or more implantable devices is further based a detection signal detected by the one or more implantable devices in the second set of one or more implantable devices. In some embodiments of the method, a determination of whether to emit the one or more electrical pulses from the one or more implantable devices in the second set of one or more implantable devices is made by an implantable device in the first set of one or implantable devices. In some embodiments of the method, a determination of whether to emit the one or more electrical pulses from the one or more implantable devices in the second set of one or more implantable devices is made by an implantable device in the second set of one or implantable devices.

In some embodiments, the method comprises directly transmitting the information related to the detection signal detected by the one or more implantable devices in the first set of one or more implantable devices from one or more of the implantable devices in the first set of one or more implantable devices to one or more of the implantable devices in the second set of one or more implantable devices.

In some embodiments, the method comprises transmitting the information related to the detection signal detected by the one or more implantable devices in the first set of one or more implantable devices from one or more of the implantable devices in the first set of one or more implantable devices to one or more of the implantable devices in the second set of one or more implantable devices through one or more intermediate devices. In some embodiments, a determination of whether to emit the one or more electrical pulses from the one or more implantable devices in the second set of one or more implantable devices is made by the one or more intermediate devices.

In some embodiments of the method, the first set of one or more implantable devices comprises two or more implantable devices. In some embodiments of the method, the second set of one or more implantable devices comprises two or more implantable devices.

In some embodiments, the method comprises generating a stimulation signal based on at least the received information related to the detection signal, wherein the stimulation signal drives the one or more electrical pulses emitted by the one or more implantable devices.

In some embodiments of the method, the detection signal comprises the one or more physiological conditions. In some embodiments, the one or more physiological conditions comprises a temperature, a respiratory rate, a strain, a pressure, a pH, a presence of an analyte, or an analyte concentration. In some embodiments of the method, the detection signal comprises the one or more electrophysiological signals.

In some embodiments of the method, the information related to the detection signal comprises: a timestamp of the electrophysiological signal or the physiological condition; or a direction, a velocity, a frequency, an amplitude, or a waveform of a compound action potential or a portion thereof within the electrophysiological signal.

In some embodiments of the method, one of the one or more implantable devices in the first set of one or more implantable devices detects the electrophysiological signal from a first nerve locus; and one of the one or more implantable devices in the second set of one or more implantable devices emits the electrical pulse configured to modulate neural activity of a second nerve locus, wherein the first nerve locus and the second nerve locus are different positions on the same nerve or different nerves. In some embodiments, the first nerve locus and the second locus are different nerves connected through a nerve network. In some embodiments, the first nerve locus and the second nerve locus are the same nerve. In some embodiments, the electrophysiological signal detected by the one of the one or more implantable devices in the first set of one or more implantable devices is transmitted by a subset of nerve fibers within the first nerve locus. In some embodiments, the subset of nerve fibers comprises one or more fascicles within the first nerve locus. In some embodiments, the subset of nerve fibers comprises one or more afferent nerve fibers. In some embodiments, the subset of nerve fibers comprises one or more efferent nerve fibers. In some embodiments, the subset of nerve fibers comprises two or more nerve fibers in different fascicles within the nerve.

In some embodiments of the method, wirelessly transmitting the information related to the detection signal from the one or more implantable devices in the first set of one or more implantable devices comprises actively transmitting from the one or more implantable devices in the first set of one or more implantable devices ultrasonic waves that encode the information related to the detection signal.

In some embodiments of the method, wirelessly transmitting the information related to the detection signal from the one or more implantable devices in the first set of one or more implantable devices comprises: receiving ultrasonic waves at the one or more implantable devices in the first set of one or more implantable devices; and backscattering the ultrasonic waves from the one or more implantable devices in the first set of one or more implantable devices, wherein the backscattered ultrasonic waves encode the information related to the detection signal.

In some embodiments of the method, the information related to the detection signal received at the one or more implantable devices in the second set of one or more implantable devices is encoded in ultrasonic waves received by the one or more implantable devices in the second set of one or more implantable devices. In some embodiments of the method, wirelessly transmitting, from the one or more implantable devices in the first set of one or more implantable devices, the information related to the detection signal detected by the one or more implantable devices in the first set of one or more implantable devices comprises: receiving, at an intermediate device, the ultrasonic waves encoding the information related to the detection signal actively transmitted or backscattered by the one or more implantable devices in the first set of one or more implantable device; actively transmitting, from the intermediate device, additional ultrasonic waves that encode the information related to the detection signal; and receiving, at the one or more implantable devices in the second set of one or more implantable devices, the additional ultrasonic waves actively transmitted from the intermediate device. In some embodiments, the intermediate device is an external device.

In some embodiments of the method, the one or more implantable devices in the first set of one or more implantable devices or the one or more implantable devices in the second set of one or more implantable devices are powered using powering ultrasonic waves. In some embodiments, the powering ultrasonic waves are transmitted by an intermediate device.

In some embodiments of the method, the electrical pulse emitted by the one or more implantable devices in the second set of one or more implantable devices is emitted to a targeted subset of nerve fibers within the target nerve. In some embodiments, the targeted subset of nerve fibers comprises one or more fascicles within the first nerve. In some embodiments, the targeted subset of nerve fibers comprises one or more afferent nerve fibers. In some embodiments, the targeted subset of nerve fibers comprises one or more efferent nerve fibers. In some embodiments, the targeted subset of nerve fibers comprises two or more nerve fibers in different fascicles within the target nerve.

In some embodiments of the method, the one or more implantable devices in the second set of one or more implantable devices emits the electrical pulse to a fibrous tissue comprising the target nerve.

In some embodiments of the method, the target nerve is a vagus nerve, a spinal cord, a splenic nerve, a mesenteric nerve, a sciatic nerve, a tibial nerve, a celiac ganglion, a sacral nerve, a renal nerve, an occipital nerve, or an adrenal nerve. In some embodiments of the method, the target nerve is a peripheral nerve.

In some embodiments of the method, the recorded nerve is a vagus nerve, a spinal cord, a splenic nerve, a mesenteric nerve, a sciatic nerve, a tibial nerve, a celiac ganglion, a sacral nerve, a renal nerve, an occipital nerve, or an adrenal nerve. In some embodiments, the recorded nerve is a peripheral nerve.

Also described herein is a device network for modulating neural activity of a target nerve, comprising: (a) one or more implantable devices in a first set of one or more implantable devices, comprising: a sensor for detecting a detection signal, comprising an electrophysiological signal transmitted by a nerve or a physiological condition, an ultrasonic transducer configured to actively transmit or backscatter ultrasonic waves, wherein the ultrasonic waves encode information related to the detection signal, and a control circuit electrically coupled to the sensor and the ultrasonic transducer; and (b) one or more implantable devices in a second set of one or more implantable devices, comprising: a plurality of electrodes configured to emit an electrical pulse to a target nerve, an ultrasonic transducer configured to receive ultrasonic waves encoding information related to the detection signal, and a control circuit configured to extract the information related to the detection signal from the ultrasonic waves, and to operate the plurality of electrodes to emit the electrical pulse based on the information related to the detection signal; wherein the one or more implantable devices in the first set of one or more implantable devices and the one or more implantable devices in the second set of one or more implantable devices are configured to wirelessly transmit information from the one or more implantable devices in the first set of one or more implantable devices to the one or more implantable devices in the second set of one or more implantable devices.

In some embodiments of the device network, the one or more implantable devices in the first set of one or more implantable devices and the one or more implantable devices in the second set of one or more implantable devices are configured to wirelessly transmit information from the one or more implantable devices in the second set of one or more implantable devices to the one or more implantable devices in the first set of one or more implantable devices.

In some embodiments, the device network comprises two or more implantable devices in the first set of one or more implantable devices.

In some embodiments, the device network comprises two or more implantable devices in the second set of one or more implantable devices.

In some embodiments of the device network, the control circuit of the one or more implantable devices in the first set of one or more implantable devices or the second set of one or more implantable devices is configured to determine whether to emit one or more electrical pulses from the one or more implantable devices based at least on information wirelessly received by the one or more implantable devices. In some embodiments, the control circuit of the one or more implantable devices in the first set of one or more implantable devices or the second set of one or more implantable devices is configured to select one or more pulse characteristics of the one or more electrical pulses. In some embodiments, determining whether to emit an electrical pulse comprises updating a dynamic state of the one or more implantable devices.

In some embodiments of the device network, the one or more implantable devices in the second set of one or more implantable devices further comprise a sensor for detecting an electrophysiological signal transmitted by a nerve or a physiological condition.

In some embodiments, the device network further comprises one or more intermediate devices comprising an ultrasonic transducer, wherein the one or more intermediate devices are configured to: wirelessly receive the information from the one or more implantable devices in the first set of one or more implantable devices through ultrasonic waves, and wirelessly transmit the information to the one or more implantable devices in the second set of one or more implantable devices through ultrasonic waves. In some embodiments, the one or more intermediate devices are further configured to: wirelessly receive the information from the one or more implantable devices in the second set one or more implantable devices of implantable devices through ultrasonic waves, and wirelessly transmit the information to the one or more implantable devices in the first set of one or more implantable devices through ultrasonic waves. In some embodiments, the one or more intermediate devices are configured to: actively transmit ultrasonic waves to the one or more implantable device in the first set of one or more implantable devices; receive backscattered ultrasonic waves that encode the information related to the detection signal detected by the sensor; and actively transmit ultrasonic waves that encode the information related to the detection signal to the one or more implantable devices in the second set of one or more implantable devices.

In some embodiments of the device network, the intermediate device comprises a control circuit configured to: extract the information related to the detection signal from the ultrasonic waves received by the intermediate device, and determine whether one or more electrical pulses should be emitted from the one or more implantable devices in the second set of one or more implantable devices based at least on information wirelessly received by the one or more implantable devices in the first set of one or more implantable devices; wherein the information related to the detection signal encoded in the ultrasonic waves transmitted from the intermediate device to the one or more implantable devices in the second set of one or more implantable devices comprises instructions to emit the one or more electrical pulses, and wherein the control circuit of the one or more implantable devices in the second set of one or more implantable devices is configured to operate the plurality of electrodes to emit the electrical pulse based on the instructions. In some embodiments, the instructions to emit the one or more electrical pulses comprise instructions for one or more pulse characteristics of the one or more electrical pulses.

In some embodiments of the device network, the control circuit of the one or more implantable devices in the first set of one or more implantable devices is configured to determine whether one or more electrical pulses should be emitted from one or more implantable devices in the second set of one or more implantable devices based on at least the detection signal detected by the sensor of the one or more implantable devices in the first set of one or more implantable devices; wherein the information related to the detection signal encoded in the ultrasonic waves actively transmitted or backscattered by the ultrasonic transducer of the one or more implantable devices in the first set of one or more implantable devices comprises instructions to emit the one or more electrical pulses; and wherein the control circuit of the one or more implantable devices in the second set of one or more implantable devices is configured to operate the plurality of electrodes to emit the electrical pulse based on the instructions. In some embodiments, the instructions to emit the one or more electrical pulses comprise instructions for one or more pulse characteristics of the one or more electrical pulses.

In some embodiments of the device network, the control circuit of the one or more implantable devices in the second set of one or more implantable devices is configured to determine whether one or more electrical pulses should be emitted from the one or more implantable devices in the second set of one or more implantable devices based on at least the information related to the detection signal, and operate the plurality of electrodes to emit the electrical pulse based on the determination. In some embodiments, the control circuit is further configured to select one or more pulse characteristics of the one or more electrical pulses.

In some embodiments of the device network, the sensor comprises a plurality of electrodes configured to detect the electrophysiological signal. In some embodiments, the sensor is configured to detect the physiological condition. In some embodiments, the physiological condition is a temperature, a respiratory rate, a strain, a pressure, a pH, a presence of an analyte, or an analyte concentration.

In some embodiments of the device network, the one or more implantable devices in the first set of one or more implantable devices comprises a first sensor configured to detect the physiological condition, and a second sensor comprising a plurality of electrodes configured to detect the electrophysiological signal.

In some embodiments of the device network, the ultrasonic transducer of the one or more implantable devices in the first set of one or more implantable devices or the ultrasonic transducer of the one or more implantable devices in the second set of one or more implantable devices is configured to receive ultrasonic waves that power the one or more implantable devices.

### BRIEF DESCRIPTION OF THE DRAWINGS

FIG. 1A shows an exemplary network with a first implantable device that directly communicates with a second implantable device. Informational flow can be unidirectional or bidirectional.

FIG. 1B shows an example a network with a first implantable device that wirelessly communicates with a second implantable device, with bidirectional communication through a recurrent neural network.

FIG. 2A illustrates an embodiment of an implantable device network that includes first implantable device, an intermediate device (which may be implanted or external), and second implantable device. The first device wirelessly communicates information to the intermediate device, which wirelessly communicates information to the second device.

FIG. 2B illustrates an embodiment of an implantable device network that includes a first implantable device, an intermediate device (which may be external or implantable), and a second implantable device. The first device wirelessly communicates information to the intermediate device, which wirelessly communicates information to the second device. The second device can then wirelessly respond to the intermediate device with additional information, which is optionally wirelessly transmitted to the first device.

FIG. 2C shows a device network configured according to a recurrent neural network, with includes a first implantable device, an intermediate device (which may be external or implantable), and a second implantable device. The first device wirelessly communicates information to the intermediate device, which wirelessly communicates information to the second device. The second device can then wirelessly respond to the intermediate device with additional information, which is wirelessly transmitted to the first device.

FIG. 3A illustrates a network with two or more implantable devices that each wirelessly communication information to another implantable device, which can optionally send information back to one or more of the original implantable devices.

FIG. 3B shows a device network configured according to a recurrent neural network, with includes a first set of implantable devices and a second set of implantable devices, which are configured to wirelessly communicate through bidirectional communication.

FIG. 4A illustrates a device network with a set of two or more implantable devices, an intermediate device, and another implantable device. The set of implantable devices wirelessly communicate information to the intermediate device, which wirelessly communicates information to the other implantable device.

FIG. 4B shows a device network configured according to a recurrent neural network, with includes a first set of implantable devices, an intermediate device, and a second set of implantable devices (which includes a single implantable device as illustrated, although could include additional implantable devices), which are configured to wirelessly communicate through bidirectional communication.

FIG. 5A illustrates a device network with a first set of two or more implantable devices and a second set of two or more implantable devices. The implantable devices in the first set wirelessly communicate information to the implantable devices in the second set. Optionally, one or more of the implantable devices in the second set wirelessly communicate additional information to the one or more implantable devices in the first set.

FIG. 5B shows a device network configured according to a recurrent neural network, with includes a first set of implantable devices and a second set of implantable devices, which are configured to wirelessly communicate through bidirectional communication.

FIG. 6A illustrates a device network with a first set of two or more implantable devices, an intermediate device, and a second set of two or more implantable devices. The first set of implantable devices wirelessly communicates information to the intermediate device, which wirelessly communicates information to the second set of implantable devices.

FIG. 6B shows a device network configured according to a recurrent neural network, with includes a first set of implantable devices and a second set of implantable devices, which are configured to wirelessly communicate through an intermediate device using bidirectional communication.

FIG. 7 illustrates a schematic of an exemplary body for the implantable device described herein, which can include an ultrasonic transducer, a battery and/or power circuit, a modulation circuit, a control circuit, a detection and/or stimulation circuit, and a non-volatile memory.

FIG. 8 illustrates a schematic of an exemplary intermediate device, which can wirelessly communicate with one or more implantable devices from a first set and/or one or more implantable devices from a second set through ultrasonic waves. The intermediate device actively transmits ultrasonic waves, and the implantable device(s) can backscatter the ultrasonic waves and encode information in the ultrasonic backscatter waves.

FIG. 9A shows a first implantable device in wireless communication with a second implantable device through ultrasonic waves.

FIG. 9B shows an implantable device in wireless communication with an intermediate device through ultrasonic waves.

FIG. 10 illustrates an exemplary process for emitting an electrical pulse based on a single component or multi-component detection signal. The detection signal (D) is analyzed to generate a trigger signal (T) obtained by the one or more implantable devices. Using the trigger signal, the implantable device can obtain a stimulation signal (S), which is used to operate a stimulation signal to emit an electrical pulse (P) to a target nerve, thereby modulating neural activity of the target nerve.

### DETAILED DESCRIPTION

Described herein are implantable devices and networks of implantable devices for modulating neural activity. Further described are methods of using such implantable device and networks of implantable devices for modulating neural activity. The network of implantable devices includes a first set of one or more implantable devices, which detect a detection signal (e.g., one or more electrophysiological signals and/or one or more physiological conditions) and a second set of one or more implantable devices, which can emit one or more electrical pulses configured to modulate neural activity of a nerve based on information related to the detection signal. In some embodiments of the network, one or more of the implantable devices in the second set can be configured to both detect a detection signal, wherein information related to the detection signal is transmitted to a separate implantable device, and receive information related to a second detection signal and emit an electrical pulse based on the information related to the second detection signal. Information related to the detection signal and/or the electrical pulse emitted by the second set of implantable devices can optionally be wirelessly transmitted back to the intermediate device or to one or more implantable devices in the first set. Once the information is received by the one or more implantable devices in the first set, the implantable device in the first set may emit another electrical pulse based on this additional information. The network can continue to operate to modulate neural activity by one or more nerves in the subject.

Implantable devices can be implanted in different locations throughout the subject. By transmitting information related to the detection signal to a different implantable device and emitting an electrical pulse based on that information, an electrical pulse can be used to modulate neural activity at a location based at least on information (e.g., a detection signal) detected at one or more different locations. This network is particularly useful because modulation of neural activity by certain nerves can have systemic or distal effects, and systemic or distal conditions can affect how a nerve should be modulated to obtain a desired effect.

The networks described herein may operate in a feedforward neural network configuration, or a recurrent neural network configuration (e.g., a Hopfield network). In a feedforward neural network, information (e.g., a detection signal) based on a first set of implantable devices is transmitted (either directly or indirectly, e.g., through one or more intermediate devices) to a second set of implantable devices that emit an electrical pulse configured to modulated neural activity based at least on the information related to the detection signal. The second set of implantable devices may further supplement the information used to emit the electrical pulse, for example with information related to an additional detection signal (which may be detected by one or more of the implantable devices in the second set) or information related to one or more previously emitted electrical pulses (which may have been emitted by one or more of the implantable devices in the second set). For example, in a feedforward configuration, an implantable device (e.g., an “output” implantable device) can emit an electrical pulse based on information detected by a separate implantable device (e.g., an “input” implantable device), and optionally information detected by the same implantable device (the “output” implantable device).

A network configured in a recurrent neural network (e.g., a Hopfield network) includes bidirectional information transfer between the two sets of implantable devices. Information related to a detection signal detected by a first set of implantable devices is wirelessly transmitted (either directly or indirectly) to a second set of implantable devices. One or more of the implantable devices in the second set may (but need not) emit an electrical pulse based on the information related to the detection signal detected by the first set of implantable devices. At least one of the implantable devices in the second set can wirelessly transmit (either directly or indirectly) additional information from the at least one implantable device in the second set to one or more implantable devices in the first set, and the one or more implantable devices may (but need not) emit an electrical pulse based on the received additional information. Such additional information can include a detection signal detected by the second set of implantable devices, or information related to a trigger signal or emitted electrical pulse. The implantable devices can include a dynamic state that is updated based on received information. Thus, a state of an implantable device in the first set can be updated based on the information received from the second set of implantable devices, and may also be updated based on information detected by the implantable device or received from one or more other implantable devices in the first set. Based on the updated dynamic state, the implantable device may emit an electrical pulse that modulates neural activity of a nerve. The other implantable devices in the network (i.e., the implantable devices in the first set and/or the second set) can be updated, and electrical pulse may be emitted based on the updated state.

The two or more implantable devices in the network can wirelessly transmit or receive the information related to the detection signal. The information may be directly communicated from one or more implantable devices in a first set to one or more implantable devices in a second set, or the information may be communicated through one or more intermediate devices, which may be implanted or external. The one or more intermediate devices can act as a relay to propagate the information (referred to as a “relay” device), or can analyze received information and transmit the analyzed information.

The one or more implantable devices wirelessly receives the information related to the detection signal (which is optionally pre-analyzed by the one or more implantable device from a first set and/or the one or more intermediate devices), and the device can emit one or more electrical pulses configured to modulate neural activity of a nerve based on the information related to the detection signal. In some embodiments, the one or more implantable devices analyze the received information related to the detection signal. Analyzing the information related to the detection signal can include generating a trigger signal based on the detection signal. The trigger signal can indicate that a stimulation signal should be generated (or one or more characteristics of the stimulation signal that should be generated), which drives the electrical pulse. Or the trigger signal may be a null signal, which indicates that no electrical pulse should be emitted. In some embodiments, the trigger signal is a dynamic state of the implantable device, which can be updated based on information received or detected by the implantable device. The information related to the detection signal can include one or more input features, such as an electrophysiological signal or a physiological condition, from one or more locations within the patient's body (with the number of locations depending on the number of implantable devices in the network). A machine learning algorithm can be used to analyze the information related to the detection signal to generate the trigger signal. The trigger signal provides instructions for if, when, or how the implantable device emits the one or more electrical pulses to modulate the neural activity of the target nerve.

The network can include N implantable devices in the first set of devices, and M implantable devices in the second set of implantable devices, wherein N and Mare one or more, and may be the same or different (although at least one implantable device is not in both sets of implantable devices). The M implantable devices can emit the one or more electrical pulses based on the detection signal detected by the N (or fewer) implantable devices. For example, in some embodiments, 1, 2, 3, 4, 5, or more implantable devices in the first set can each detect a component of the detection signal, and information related to the detection signal from the implantable devices in the first set is wirelessly transmitted to 1, 2, 3, 4, 5, or more or more implantable devices in the second set, and one or more of the implantable devices in the second set can emit one or more electrical pulses.

In some embodiments, a network for modulating neural activity comprises (a) one or more implantable devices in a first set of one or more implantable devices, comprising: a sensor for detecting a detection signal comprising an electrophysiological signal transmitted by a nerve or a physiological condition, an ultrasonic transducer configured to actively transmit or backscatter ultrasonic waves, wherein the ultrasonic waves encode information related to the detection signal, and a control circuit electrically coupled to the sensor and the ultrasonic transducer; and (b) one or more implantable devices in a second set of one or more implantable devices, comprising: a plurality of electrodes configured to emit an electrical pulse to a target nerve, an ultrasonic transducer configured to receive ultrasonic waves encoding information related to the detection signal, and a control circuit configured to extract the information related to the detection signal from the ultrasonic waves, and to operate the plurality of electrodes to emit the electrical pulse based on the information related to the detection signal; wherein the one or more implantable devices in the first set of one or more implantable devices and the one or more implantable devices in the second set of one or more implantable devices are configured to wirelessly transmit information from the one or more implantable devices in the first set of one or more implantable devices to the one or more implantable devices in the second set of one or more implantable devices.

Information can be wirelessly transmitted or received through any suitable technique, such as ultrasonic communication. Exemplary methods for ultrasonic communication are described in US 2018/0085605; WO 2018/009908; WO 2018/009910; WO 2018/009911; and WO 2018/009912. For example, the implantable devices can transmit information by actively transmitting (i.e., generating) ultrasonic waves that encode the information that are received by another device, or by backscattering ultrasonic waves received by the device, wherein the ultrasonic backscatter waves encode the information.

In some embodiments, a method of modulating neural activity using an implantable device network includes (a) detecting, at one or more implantable devices in a first set of one or more implantable devices, a detection signal comprising one or more electrophysiological signals transmitted by a recorded nerve or one or more physiological conditions; (b) wirelessly transmitting, from the one or more implantable devices in the first set of one or more implantable devices, information related to the detection signal; (c) wirelessly receiving, at one or more implantable devices in a second set of one or more implantable devices, the information related to the detection signal; and (d) determining whether to emit, from one or more implantable devices in the second set of one or more implantable devices, one or more electrical pulses configured to modulate neural activity of one or more target nerves based on at least the received information related to the detection signal. The method can further include emitting, at the one or more implantable devices in the second set of one or more implantable devices, the one or more electrical pulses. In some embodiments, the method further includes wirelessly transmitting, from the one or more implantable devices in the second set of one or more implantable devices, information related to the one or more implantable devices in the second set of one or more implantable devices; wirelessly receiving, at the one or more implantable devices in the first set of one or more implantable devices, the information related to the one or more implantable devices in the second set of one or more implantable devices; and determining whether to emit, from one or more implantable devices in the first set of one or more implantable devices, one or more electrical pulses configured to modulate neural activity of one or more additional target nerves based on at least the information related to the one or more implantable devices in the second set of one or more implantable devices.

### Definitions

As used herein, the singular forms “a,” “an,” and “the” include the plural reference unless the context clearly dictates otherwise.

Reference to “about” or “approximately” a value or parameter herein includes (and describes) variations that are directed to that value or parameter per se. For example, description referring to “about X” includes description of “X.”

“Actively transmitted” ultrasonic waves that are transmitted or emitted from a device refer to ultrasonic waves that are generated by and originate from that device. The device can encode information in the actively transmitted ultrasonic waves that are emitted from the device.

“Backscattering” ultrasonic waves refers to ultrasonic waves that are received by a device and reflected by that device. The reflected ultrasonic waves can be referred to as “ultrasonic backscatter waves,” and the device can encode information in the ultrasonic backscatter waves as the ultrasonic waves are reflected by the device. Information can thus be transmitted by ultrasonic backscatter waves.

It is understood that aspects and variations of the invention described herein include “consisting” and/or “consisting essentially of” aspects and variations.

The terms “implantable” and “implanted” refer to an object being fully implantable or fully implanted in a subject such that no portion of the object breaches the surface of the subject. Any device described herein as implantable can be implanted in a subject.

An “input implantable device” refers to an implantable device configured to detect a detection signal. An “output implantable device” refers to an implantable device configured to emit an electrical pulse based at least on information related to the detection signal. An output implantable device can optionally detect an additional detection signal, and the emitted electrical pulse may be based on the information related to the detection signal from one or more input devices and information related to the additional detection signal.

As used herein, the term “physiological condition” refers to a physiological state, or parameters or values, estimated or measured within a physiological environment. Accordingly, a “physiological condition” can include a temperature, pH, pO₂, heart rate, the presence of an analyte, an amount of an analyte, a strain, or any other value measured within a physiological environment.

A “set of implantable devices” refers to one or more implantable devices. A “first set of implantable devices” and “second set of implantable devices” may overlap as long as at least first set of implantable devices and the second set of implantable devices are not identical.

The term “substantially” refers to 70% or more. For example, a curved member that substantially surrounds a cross-section of a nerve refers to a curved member that surrounds 70% or more of the cross-section of the nerve.

The term “subject” and “patient” are used interchangeably herein to refer to a vertebrate animal.

The terms “treat,” “treating,” and “treatment” are used synonymously herein to refer to any action providing a benefit to a subject afflicted with a disease state or condition, including improvement in the condition through lessening, inhibition, suppression, or elimination of at least one symptom, delay in progression of the disease or condition, delay in recurrence of the disease or condition, or inhibition of the disease or condition.

Where a range of values is provided, it is to be understood that each intervening value between the upper and lower limit of that range, and any other stated or intervening value in that stated range, is encompassed within the scope of the present disclosure. Where the stated range includes upper or lower limits, ranges excluding either of those included limits are also included in the present disclosure.

It is to be understood that one, some or all of the properties of the various embodiments described herein may be combined to form other embodiments of the present invention. The section headings used herein are for organizational purposes only and are not to be construed as limiting the subject matter described.

Features and preferences described above in relation to “embodiments” are distinct preferences and are not limited only to that particular embodiment; they may be freely combined with features from other embodiments, where technically feasible, and may form preferred combinations of features. The description is presented to enable one of ordinary skill in the art to make and use the invention and is provided in the context of a patent application and its requirements. Various modifications to the described embodiments will be readily apparent to those persons skilled in the art and the generic principles herein may be applied to other embodiments. Thus, the present invention is not intended to be limited to the embodiment shown but is to be accorded the widest scope consistent with the principles and features described herein.

### Device Networks

The implantable device network includes one or more implantable devices in a first set configured to detect a detection signal comprising one or more electrophysiological signals or one or more physiological conditions, and one or more implantable devices in a second set configured to emit one or more electrical pulses configured to modulate neural activity of a nerve based at least on the information related to the detection signal. The information transmitted by the first set of implantable device may also include information related to a dynamic state of one or more implantable devices in the first set. In some embodiments, the device network can include one or more intermediate devices, which can function as a relay for information between one or more implantable devices, or can analyze and/or process the information related to the detection signal before it is transmitted to the one or more implantable devices in the second set. In some embodiments, the one or more implantable devices in the second set detect an additional detection signal comprising an electrophysiological signal and/or a physiological condition, the one or more electrical pulses emitted by the one or more implantable devices can be further based on the additional detection signal.

In some embodiments, the implantable devices are implanted in a subject. The subject can be for example, a mammal. In some embodiments, the subject is a human, dog, cat, horse, cow, pig, sheep, goat, monkey, or a rodent (such as a rat or mouse). Preferably, the subject is a human.

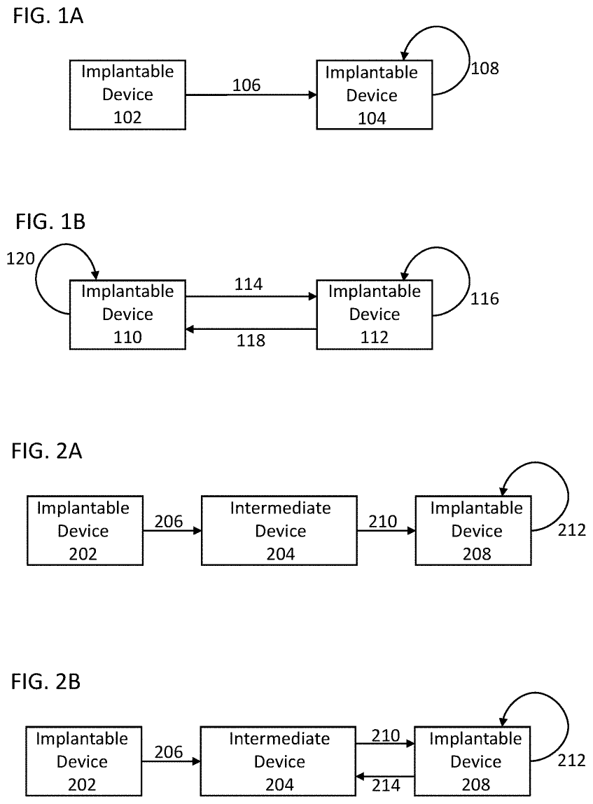

FIG. 1A illustrates an exemplary network with a first implantable device 102 that directly communicates with a second implantable device 104, as indicated by arrow 106. The first implantable device 104 detects a detection signal, and wirelessly transmits information related to the detection signal to the second implantable device 104. Optionally, the first implantable device transmits other information to the second implantable device, such as information related to a dynamic state of the first implantable device 102, a location of the first implantable device 102, or information related to an electrical pulse emitted by the first implantable device 102. Once the second implantable device 104 receives the information, the second implantable device 104 can emit one or more electrical pulses that modulate neural activity of a target nerve base at least on the information related to the detection signal, and optionally some or all of the additional information transmitted by the first implantable device 102. In some embodiments, the first implantable device 102 analyzes the detection signal to generate a trigger signal for the first implantable device 102 (e.g., a dynamic state of the first implantable device 102) or a trigger signal for the second implantable device 104, and the trigger signal is transmitted to the second implantable device 104. The second implantable device can then emit one or more electrical pulses to modulate neural activity of the nerve based on the trigger signal. In some embodiments, second implantable device 104 analyzes the information related to the detection signal (and, optionally, additional information transmitted by the first implantable device 102) to generate the trigger signal, and can emit one or more electrical pulses to modulate neural activity of the nerve based on the trigger signal. In some embodiments, the second implantable device 104 detects an additional detection signal (as indicated by arrow 108), and the second implantable device 102 analyzes the information related to the detection signal detected by the first implantable device 102 and the detection signal detected by the second implantable device 104 to generate the trigger signal.

FIG. 1B shows an example a network with a first implantable device 110 that wirelessly communicates with a second implantable device 112, with bidirectional communication through a recurrent neural network. The first implantable device 110 detects a detection signal, and wirelessly transmits information related to the detection signal to the second implantable device 112, as indicated by arrow 114. Optionally, the first implantable device transmits other information to the second implantable device, such as information related to a dynamic state of the first implantable device 110, a location of the first implantable device 110, or information related to an electrical pulse emitted by the first implantable device 110. Once the second implantable device 112 receives the information, the second implantable device 112 can emit one or more electrical pulses that modulate neural activity of a target nerve base at least on the information related to the detection signal, and optionally some or all of the additional information transmitted by the first implantable device 110. In some embodiments, the first implantable device 110 analyzes the detection signal to generate a trigger signal for the second implantable device 112, and the trigger signal is transmitted to the second implantable device 112. The second implantable device 112 can then emit one or more electrical pulses to modulate neural activity of the nerve based on the trigger signal. In some embodiments, second implantable device 112 analyzes the information related to the detection signal (and, optionally, additional information transmitted by the first implantable device 110) to generate the trigger signal, and can emit one or more electrical pulses to modulate neural activity of the nerve based on the trigger signal. In some embodiments, the second implantable device 112 detects an additional detection signal (as indicated by arrow 116), and the second implantable device 112 analyzes the information related to the detection signal detected by the first implantable device 112 and the detection signal detected by the second implantable device 114 to generate the trigger signal (e.g., a dynamic state) for the second implantable device 112. The second implantable device can then emit an electrical pulse based on the trigger signal, or not (based on the trigger signal).

The second implantable device 112 can wirelessly transmit information back to the first implantable device 110, as indicated by arrow 118. The information transmitted by the second implantable device 112 to the first implantable device 110 can include a detection signal detected by the second implantable device, information related to an electrical pulse emitted by the second implantable device 112, or information related to the dynamic state of the second implantable device (for example, a trigger signal generated by the second implantable device 112). As the dynamic state of the second implantable device 112 be updated based on the information received from the first implantable device 112 and/or the detection signal detected by the second implantable device 112, so too can the dynamic state of the first implantable device 110 be updated based on the information received from the second implantable device. The dynamic state of the first implantable device 110 can also or alternatively be updated based on a detection signal detected by the first implantable device 110 (which may be the same detection signal information transmitted to the second implantable device as previously discussed, or a new detection signal detected by the first implantable device 110), as indicated by arrow 120. Once the dynamic state of the first implantable device 110 has been updated (i.e., a trigger signal has been generated for the first implantable device), the first implantable device 110 can emit an electrical pulse to modulate neural activity (or not) based on the trigger signal, if the first implantable device is configured to do so.

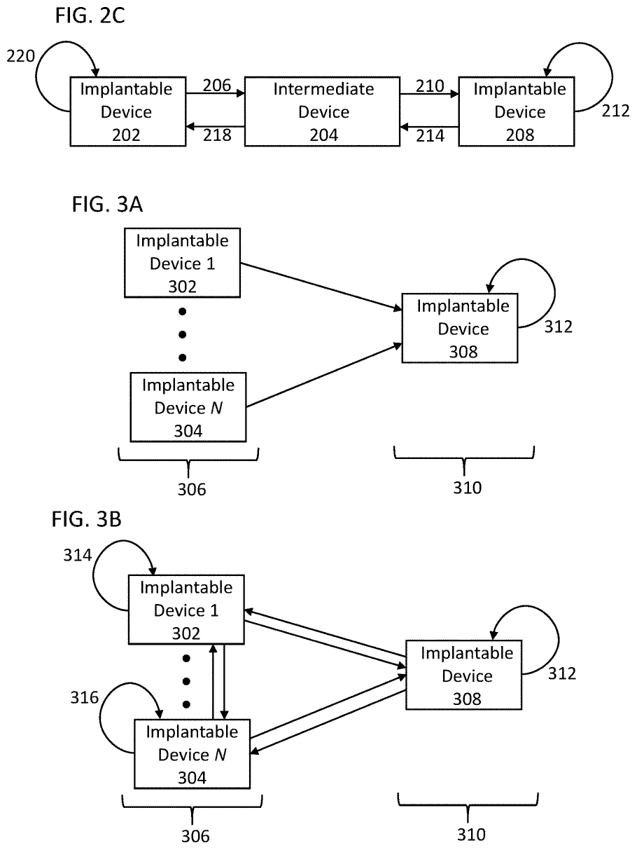

The first implantable device and the second implantable device can communicate directly, as indicated in FIGS. 1A-1B, or can communicate through one or more intermediate devices, as indicated in FIGS. 2A-2B. The intermediate device can be the same type of device as the first or second implantable devices, or can be a different type of device, as long as the intermediate device is functionally configured as described herein. FIG. 2A illustrates an embodiment of an implantable device network that includes a first implantable device 202, an intermediate device 204, and a second implantable device 206. The first implantable device 202 detects a detection signal and wirelessly transmits information related to the detection signal to the intermediate device 204, as indicated by arrow 206. The information transmitted by the first implantable device 202 may include additional information, such as a dynamic state of the first implantable device 202, a location of the first implantable device 202, or information about an electrical pulse emitted by the first implantable device 202. The intermediate device 204 receives the information related to the detection signal from the first implantable device 202 (and, optionally, other information transmitted by the first implantable device) and transmits the information related to the detection signal to the second implantable device 208, as indicated by arrow 210. Once the second implantable device 208 receives the information related to the detection signal, the second implantable device 208 can emit one or more electrical pulses that modulate neural activity of a target nerve based on the information. Although FIG. 2A illustrates a single intermediate device 204, it is contemplated that one or more (e.g., 2, 3, 4, or more) intermediate devices can be used by the network. That is, a first intermediate device can receive the information related to the detection signal, and the first intermediate device can transmit the information related to the detection signal to a second intermediate device. The second intermediate device can then transmit the information to the implantable device in the second set, or to a third intermediate device. In some embodiments, the first implantable device 202 analyzes the detection signal to generate a trigger signal for the second implantable device 208, which is transmitted to the intermediate device 204. The intermediate device 204 can then function as a relay to transmit the trigger signal received from the first implantable device 202 to the second implantable device 208. Once the second implantable device 208 receives the trigger signal, the second implantable device 208 can emit the one or more electrical pulses that modulate neural activity of a target nerve (or not, if the trigger signal is a null signal). In some embodiments, the first implantable device 202 wirelessly transmits information related to the detection signal (and, optionally, additional information about the first implantable device 202, such as a dynamic state of the device), which is received by the intermediate device 204. The intermediate device 204 can then wirelessly relay the information to the second implantable device 208, which analyzes the information related to the detection signal to generate a trigger signal. The second implantable device 208 can then emit one or more electrical pulses that modulate neural activity of a target nerve based on the trigger signal. Optionally, the second implantable device 208 detects an additional detection signal (as indicated by arrow 212), and the second implantable device 208 analyzes the information related to the detection signal detected by the first implantable device 202 and the detection signal detected by the second implantable device 208 to generate the trigger signal. In some embodiments, the first implantable device 202 wirelessly transmits information including the information related to the detection signal to the intermediate device 204, which analyzes the information to generate a trigger signal for the second implantable device 208.

Optionally, the second implantable device 208 detects an additional detection signal, and information related to the additional detection signal is wirelessly transmitted to the intermediate device 204 as indicated by arrow 214 in FIG. 2B, and the intermediate device 204 analyzes the information related to the detection signal detected by the first implantable device 202 and the information related to the additional detection signal detected by the second implantable device 208 to generate a trigger signal (see FIG. 2B). The intermediate device 204 then wirelessly transmits the trigger signal for the second implantable device 208 to the second implantable device 208, which emits one or more electrical pulses that modulates neural activity of a target nerve based on the trigger signal.

FIG. 2C shows a device network configured according to a recurrent neural network, with includes a first implantable device, a second implantable device, and an intermediate device that are configured to wirelessly communicate through bidirectional communication. The first implantable device 202 detects a detection signal, and wirelessly transmits information related to the detection signal to the intermediate device 204, as indicated by arrow 204. Optionally, the first implantable device transmits other information to the intermediate device 204, such as information related to a dynamic state of the first implantable device 202, a location of the first implantable device 202, or information related to an electrical pulse emitted by the first implantable device 202. The intermediate device 204, once it receives the information from the first implantable device 202, wirelessly transmits the information to the second implantable device 208, as indicated by arrow 210. Once the second implantable device 208 receives the information, the second implantable device 208 can emit one or more electrical pulses that modulate neural activity of a target nerve base at least on the information related to the detection signal, and optionally some or all of the additional information transmitted by the first implantable device 202.

Still referring to FIG. 2C, in some embodiments, the first implantable device 202 analyzes the detection signal to generate a trigger signal for the second implantable device 208, and the trigger signal is transmitted to the second implantable device 208 through the intermediate device 204. The second implantable device 208 can then emit one or more electrical pulses to modulate neural activity of the nerve based on the trigger signal. In another embodiment, the intermediate device 204 receives the information from the first implantable device 202, and generates the trigger signal for the second implantable device 208. The intermediate device 204 can then wirelessly transmit the trigger signal to the second implantable device 208, which emits the electrical pulse based on the received trigger signal.

In some embodiments, second implantable device 208 analyzes the information related to the detection signal (and, optionally, additional information transmitted by the first implantable device 202) to generate the trigger signal, and can emit one or more electrical pulses to modulate neural activity of the nerve based on the trigger signal generated by the second implantable device. The trigger signal can be the dynamic state of the second implantable device, which is updated based on the received information. In some embodiments, the second implantable device 208 detects an additional detection signal (as indicated by arrow 212), and the second implantable device 208 analyzes the information related to the detection signal detected by the first implantable device 202 and the detection signal detected by the second implantable device 208 to generate the trigger signal (e.g., a dynamic state) for the second implantable device 208. The second implantable device can then emit an electrical pulse based on the trigger signal, or not (based on the trigger signal).

The second implantable device 208 can wirelessly transmit information back to the first implantable device 202 through the intermediate device 204, as indicated by arrow 214 and arrow 218. The information transmitted by the second implantable device 208 to the first implantable device 202 can include a detection signal detected by the second implantable device, information related to an electrical pulse emitted by the second implantable device 208, or information related to the dynamic state of the second implantable device 208 (for example, a trigger signal generated by the second implantable device 208). As the dynamic state of the second implantable device 208 be updated based on the information received from the first implantable device 202 and/or the detection signal detected by the second implantable device 208, so too can the dynamic state of the first implantable device 202 be updated based on the information received from the second implantable device 208. The dynamic state of the first implantable device 202 can also or alternatively be updated based on a detection signal detected by the first implantable device 202 (which may be the same detection signal information transmitted to the second implantable device as previously discussed, or a new detection signal detected by the first implantable device 202), as indicated by arrow 220. Once the dynamic state of the first implantable device 202 has been updated (i.e., a trigger signal has been generated for the first implantable device), the first implantable device 202 can emit an electrical pulse to modulate neural activity (or not) based on the trigger signal, if the first implantable device is configured to do so.

FIG. 3A illustrates a network with two or more implantable devices (e.g., 302 and 304) in a first set 306 and another implantable device 308 in a second set 310. Implantable device 304 is shown as “Implantable Device N” to indicate that any number of implantable devices can be included in the network, wherein N is 2 or more (e.g., 2, 3, 4, 5, 6, 7, 8, 9, 10, or more). For simplification, the following describes the network in reference to two implantable devices in the first set of implantable devices and a single implantable device in the second set, but is understood that the number of implantable devices in the sets can be expanded. The first implantable device 302 can detect a first detection signal, and the second implantable device 304 can detect a second detection signal. Information related to the first and second detection signals (or, collectively, the detection signal) are wirelessly transmitted to the other implantable device 308 in the second set. Optionally, additional information, such as a dynamic state of the first or second implantable device, a location of the first or second implantable device, and/or information related to one or more electrical pulses emitted by the first or second implantable device in the first set 306 is wirelessly transmitted to the implantable device 308 in the second set 310. The implantable device 308 in the second set 310 analyzes the detection signal received from the first implantable device 302 and the second implantable device 304 in the first set 306 to generate a trigger signal (e.g., to update a status of the implantable device), and the implantable device 308 can emit one or more electrical pulses that modulate neural activity of a target nerve based on the trigger signal. Optionally, the implantable device 308 in the second set 310 detects an additional detection signal as indicated by arrow 312, and the implantable device 308 analyzes the information related to the detection signal detected by the two or more implantable devices from the first set 308 and the detection signal detected by the implantable device 308 of the second set to generate the trigger signal.

FIG. 3B shows a device network configured according to a recurrent neural network, with includes a first set of implantable devices and a second set of implantable devices (which includes a single implantable device as illustrated, although could include additional implantable devices), which are configured to wirelessly communicate through bidirectional communication. The implantable devices 302 and 304 in the first set 306 wirelessly transmit information related to a detection signal to the implantable device 308 in the second set 310. Optionally, the implantable device 302 and 304 wirelessly transmit additional information about one or more of the implantable devices in the first set 306, such as a dynamic state of one or more of the implantable devices, a location of one or more of the implantable devices, or an electrical pulse emitted by one or more of the implantable devices. Once the implantable device 308 in the second set 310 receives the information, the implantable device 308 can emit one or more electrical pulses that modulate neural activity of a target nerve base at least on the information related to the detection signal, and optionally some or all of the additional information transmitted by the implantable devices in the first set 306. The implantable device 308 in the second set 308 analyzes the information related to the detection signal (and, optionally, some or all of the additional information transmitted by the first set 306 of implantable devices) to generate the trigger signal (e.g., update a dynamic status of the device), and can emit one or more electrical pulses to modulate neural activity of the nerve based on the trigger signal (or not, if the trigger signal is a null signal). In some embodiments, the implantable device 308 of the second set 310 detects an additional detection signal (as indicated by arrow 312), and the implantable device 308 analyzes the information related to the detection signal detected by the first set 306 of implantable devices (and optionally the additional information transmitted by the first set 306 of devices) and the detection signal detected by the implantable device 308 of the second set to generate the trigger signal (e.g., update a dynamic state of the device) for the implantable device 308 of the second set 310. The implantable device 308 can then emit an electrical pulse based on the trigger signal, or not (based on the trigger signal).

The implantable device 308 can wirelessly transmit information back to the implantable devices 302 and/or 304 in the first set 306. The information transmitted by the implantable device 308 of the second set 310 to the second set 306 of implantable devices can include a detection signal detected by the implantable device 308 in the second set 310, information related to an electrical pulse emitted by the implantable device 308 in the second set 310, or information related to the dynamic state of the implantable device 308 in the second set 310 (for example, a trigger signal generated by the implantable device). Once the implantable devices in the first set 306 receive the information from the implantable device 308 in the second set, the dynamic state of the implantable devices 302 and 304 in the first set 306 can be updated based on the received information. The dynamic state of the first implantable device 302 in the first set 306 and the second implantable device 304 in the first set 306 can also or alternatively be updated based on a detection signal detected by the first implantable device 302 or the second implantable device 304 (which may be the same detection signal information transmitted to the second implantable device as previously discussed, or a new detection signal detected by the first or second implantable devices of the first set 306), as indicated by arrow 314 and arrow 316. In some embodiments, the implantable devices within the first set 306 can wirelessly communicate between the devices (e.g., implantable device 302 and implantable device 304 can wirelessly communicate with each other) to transmit additional information, such as detection signal components or a dynamic state of the device. Once the dynamic state of the first implantable device 302 and/or second implantable device 304 in the first set 306 has been updated (i.e., a trigger signal has been generated for the first or second implantable device), the first implantable device 302 and/or second implantable device 304 can emit an electrical pulse to modulate neural activity (or not) based on the trigger signal, if the first implantable device or the second implantable device are configured to do so.

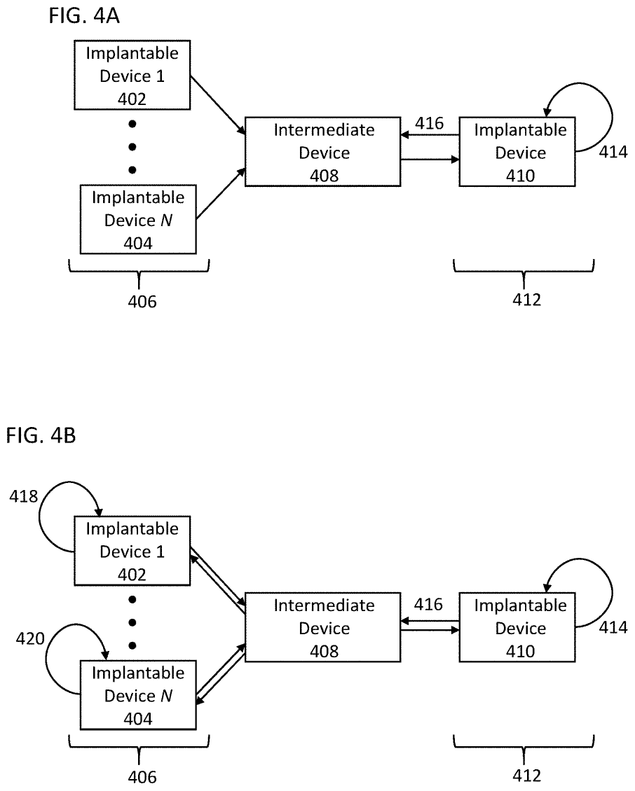

FIG. 4A illustrates a device network with two or more implantable devices (e.g. 402 and 404) in a first set 406 of implantable devices, an intermediate device 408, and an implantable device 410 in a second set 412 of implantable devices. Implantable device 404 is shown as “Implantable Device N” to indicate that any number of implantable devices can be included in the first set of the network, wherein N is 2 or more (e.g., 2, 3, 4, 5, 6, 7, 8, 9, 10, or more). For simplification, the following describes the network in reference to two implantable devices in the first set 406, but is understood that the number of implantable devices in the first set can be expanded. Additionally, although FIG. 4A illustrates a single intermediate device 408, it is contemplated that one or more (e.g., 2, 3, 4, or more) intermediate devices can be used by the network. That is, a first intermediate device can receive the information related to the detection signal, and the first intermediate device can transmit the information related to the detection signal to a second intermediate device. The second intermediate device can then transmit the information to the implantable device 410 of the second set, or to a third intermediate device. The first implantable device 402 can detect a first detection signal, and the second implantable device 404 can detect a second detection signal, and information related to the first and second detection signals (or, collectively, the detection signal) are wirelessly transmitted to the intermediate device 408. The information transmitted by the implantable devices in the first set 406 may include additional information, such as a dynamic state of one or more of the implantable devices 402 or 404 in the first set 406, a location of one or more of the implantable devices in the first set 406, or information about an electrical pulse emitted by one or more of the implantable devices in the first set 406. The intermediate device 408 then wirelessly transmits the information related to the detection signal (and, optionally, other information transmitted by the implantable devices in the first set) to the implantable device 410 in the second set 412, which emits one or more electrical pulses that modulate neural activity of a target nerve based on the received information.

In some embodiments, the intermediate device 408 wirelessly relays the information received from the two or more implantable devices (402 and 404) in the first set 406 to the implantable device 410 in the second set 412 by receiving the information from the first set 406 of implantable devices and transmitting the information without analyzing the information. The implantable device 410 in the second set 412 then receives the information, including the information related to the detection signal, and analyzes the information to generate a trigger signal (e.g., update a dynamic state of the implantable device 410). Optionally, the implantable device 410 detects an additional detection signal, and the implantable device 410 analyzes the information related to the detection signal detected by the two or more implantable devices (402 and 404) in the first set 406 (and, optionally, some or all of the additional information transmitted by the first set 406 of implantable devices) and the detection signal detected by the implantable device 410 in the second set 412 to generate the trigger signal (e.g., update the dynamic state), as indicated by arrow 414. Once the implantable device 410 generates the trigger signal, it can emit one or more electrical pulses that modulate neural activity of a target nerve (or not emit such a pulse) based on the trigger signal.

In some embodiments, the intermediate device 408 receives the information from the first set 406 of implantable devices, including the information related to the detection signal detected by the two or more implantable devices (402 and 404), analyzes the information related to the detection signal to generate a trigger signal for the implantable device 410 in the second set, and wirelessly transmits the trigger signal to the implantable device 410 in the second set 412. Once the implantable device 410 receives the trigger signal, it can emit one or more electrical pulses that modulate neural activity of a target nerve (or not emit such a pulse) based on the trigger signal.

In some embodiments, the implantable device 410 in the second set 412 detects an additional detection signal (as indicated by arrow 414), which is wirelessly transmitted to the intermediate device 408 (as indicated by arrow 416). The implantable device 410 in the second set 412 can optionally transmit additional information about the implantable device 410 in the second set 412, such as information related to the dynamic state of the implantable device 410, or a location of the implantable device 410. The intermediate device 408 analyzes the information related to the detection signal detected by the two or more implantable devices (402 and 404) in the first set 406 (and optionally some or all of the additional information transmitted by the first set 406 of implantable devices) and the information transmitted by the implantable device 410 in the second set 412, including the information related to the additional detection signal detected by the implantable device 410 in the second set 412, to generate a trigger signal for the implantable device 410 in the second set 412. Once the implantable device 410 in the second set 412 receives the trigger signal, the implantable device 410 can emit one or more electrical pulses that modulate neural activity of a target nerve based on the trigger signal (or not, if the trigger signal is a null signal).

FIG. 4B shows a device network configured according to a recurrent neural network, with includes a first set of implantable devices, an intermediate device, and a second set of implantable devices (which includes a single implantable device as illustrated, although could include additional implantable devices), which are configured to wirelessly communicate through bidirectional communication. The implantable devices 402 and 404 in the first set 406 wirelessly transmit information, including at least information related to a detection signal, to the intermediate device 408. The information transmitted by the implantable devices in the first set 406 may include additional information, such as a dynamic state of one or more of the implantable devices 402 or 404 in the first set 406, a location of one or more of the implantable devices in the first set 406, or information about an electrical pulse emitted by one or more of the implantable devices in the first set 406. The intermediate device 408 then wirelessly transmits the information related to the detection signal (and, optionally, other information transmitted by the implantable devices in the first set) to the implantable device 410 in the second set 412, which emits one or more electrical pulses that modulate neural activity of a target nerve based on the received information.

The intermediate device 408 can wirelessly relay the information received from the two or more implantable devices (402 and 404) in the first set 406 to the implantable device 410 in the second set 412 by receiving the information from the first set 406 of implantable devices and wirelessly transmitting the information to the implantable device 410 in the second set 412, either with or without analyzing the information. The implantable device 410 in the second set 412 then receives the information, including the information related to the detection signal (and optionally the additional information transmitted by the implantable devices in the first set 406), and analyzes the information to generate a trigger signal (e.g., update a dynamic state of the implantable device 410), if the information was not pre-analyzed by the intermediate device 408. Optionally, the implantable device 410 detects an additional detection signal, and the implantable device 410 analyzes the information related to the detection signal detected by the two or more implantable devices (402 and 404) in the first set 406 (and, optionally, some or all of the additional information transmitted by the first set 406 of implantable devices) and the detection signal detected by the implantable device 410 in the second set 412 to generate the trigger signal (e.g., update the dynamic state), as indicated by arrow 414. Once the implantable device 410 generates the trigger signal, it can emit one or more electrical pulses that modulate neural activity of a target nerve (or not emit such a pulse) based on the trigger signal.

The implantable device 410 of the second set 412 can wirelessly transmit information back to the intermediate device 408, which can analyze the information from the implantable device 410 and/or relay the information to the implantable devices 402 and/or 404 in the first set 406. The information transmitted by the implantable device 408 of the second set 412 to the intermediate device can include a detection signal detected by the implantable device 410 in the second set 412, information related to an electrical pulse emitted by the implantable device 410 in the second set 412, or information related to the dynamic state of the implantable device 410 in the second set 412 (for example, a trigger signal generated by the implantable device). The intermediate device 408 can analyze the received information to generate a trigger signal (e.g., an updated dynamic state) for one or more of the implantable devices in the first set 406, or the intermediate device 408 can relay the information to one or more of the implantable devices in the first set 406. Once the implantable devices in the first set 406 receive the information from the intermediate devices, the dynamic state of the implantable devices 402 and 404 in the first set 406 can be updated based on the received information. The dynamic state of the first implantable device 402 in the first set 406 and the second implantable device 404 in the first set 406 can also or alternatively be updated based on a detection signal detected by the first implantable device 402 or the second implantable device 404 (which may be the same detection signal information transmitted to the second set 412 or a new detection signal detected by the first or second implantable devices of the first set 406), as indicated by arrow 418 and arrow 420. In some embodiments, the implantable devices within the first set 406 can wirelessly communicate between the devices (e.g., implantable device 402 and implantable device 404 can wirelessly communicate with each other) to transmit additional information, such as detection signal components or a dynamic state of the device. Once the dynamic state of the first implantable device 402 and/or second implantable device 404 in the first set 406 has been updated (i.e., a trigger signal has been generated for the first implantable device), the first implantable device 402 and/or second implantable device can emit an electrical pulse to modulate neural activity (or not) based on the trigger signal, if the first implantable device or the second implantable device are configured to do so.

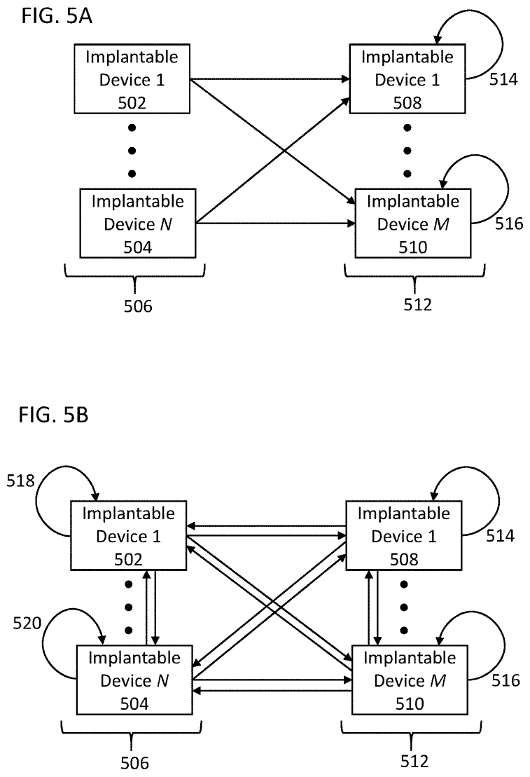

FIG. 5A illustrates a device network with two or more implantable devices (e.g., 502 and 504) in a first set 506, and two or more implantable devices (e.g., 508 and 510) in a second set, wherein the implantable devices in the first set 506 communicate directly with the implantable devices in the second set 512. Implantable device 504 is shown as “Implantable Device A” to indicate that any number of implantable devices can be included in the first set 506 of the network, wherein N is 2 or more (e.g., 2, 3, 4, 5, 6, 7, 8, 9, 10, or more). Similarly, implantable device 510 is shown as “Implantable Device M” to indicate that any number of implantable devices can be included in second set 512 of the network, wherein M is 2 or more (e.g., 2, 3, 4, 5, 6, 7, 8, 9, 10, or more). For simplification, the following describes the network in reference to two implantable devices in the first set 506, and two implantable devices in the second set 512, but is understood that the number of implantable devices in either or both sets can be expanded. The first implantable device 502 in the first set 506 detects a first detection signal, and the second implantable device 504 in the first set 506 detects a second detection signal. Information related to the first and second detection signals (collectively, the detection signals) are wirelessly transmitted from the first and second implantable devices (502 and 504), and the information is received by the first implantable device 508 of the second set 512 and the second implantable device 510 of the second set 512. The information transmitted by the implantable devices in the first set 506 can optionally include additional information, such as a dynamic state of one or more of the implantable devices 502 or 504 in the first set 506, a location of one or more of the implantable devices in the first set 506, or information about an electrical pulse emitted by one or more of the implantable devices in the first set 506. The first implantable device 508 in the second set 512 can analyze the information received by the implantable devices in the first set 506, and can generate a trigger signal (e.g., update a dynamic state) for the first implantable device 508 in the second set. The first implantable device 508 in the second set 512 can then emit (or not emit) one or more electrical pulses configured to modulate neural activity of a first nerve position based on the information related to the detection signal (i.e., based on the generated trigger signal). Similarly, the second implantable device 508 can analyze the information received from the implantable devices in the first set 506, and can generate a trigger signal (e.g., update a dynamic state) for the second implantable device 510 in the second set. The second implantable device 510 in the second set 512 can then emit (or not emit) one or more electrical pulses configured to modulate neural activity of a second nerve position based on the information related to the detection signal (i.e., based on the generated trigger signal). The electrical pulses emitted from the different implantable devices in the second set may be the same or different, even though they might receive the same information, including information related to the detection signal, from the first set 506 because the different implantable devices target different nerve positions. Optionally, the first implantable device 508 from the second set 512 detects an additional detection signal (as indicated by arrow 514), and the first implantable device 508 analyzes the information from the first set 506 of implantable devices and the information related to the detection signal detected by the first implantable device 508 of the second set 512 (and, optionally additional information, such as a dynamic state of the first implantable device 508 of the second set 512) to generate the trigger signal (e.g., to update the dynamic state of the second implantable device 508). Optionally or additionally, the second implantable device 510 detects an additional detection signal (as indicated by arrow 516), and the second implantable device 510 analyzes the information from the two or more implantable devices (502 and 504) of the first set 506, and the information related to the detection signal detected by the second implantable device 510 of the second set 512 (and, optionally additional information, such as a dynamic state of the second implantable device 510 of the second set 512) to generate the trigger signal for the implantable device 510 of the second set 512.

FIG. 5B shows a device network configured according to a recurrent neural network, with includes a first set of implantable devices and a second set of implantable devices, which are configured to wirelessly communicate through bidirectional communication. The implantable devices 502 and 504 in the first set 506 wirelessly transmit information related to a detection signal to implantable device 508 and implantable device 510 in the second set 512. Optionally, the implantable devices 502 and/or 504 wirelessly transmit additional information about one or more of the implantable devices in the first set 506, such as a dynamic state of one or more of the implantable devices, a location of one or more of the implantable devices, or an electrical pulse emitted by one or more of the implantable devices in the first set 506. Once the implantable devices in the second set 512 receives the information, the implantable devices 508 and/or 510 can emit (or not emit) one or more electrical pulses that modulate neural activity of a target nerve base at least on the information related to the detection signal, and optionally some or all of the additional information transmitted by the implantable devices in the first set 506. The implantable devices in the second set 512 analyzes the information related to the detection signal (and, optionally, some or all of the additional information transmitted by the first set 506 of implantable devices) to generate the trigger signal (e.g., update a dynamic status of the device), and can emit one or more electrical pulses to modulate neural activity of the nerve based on the trigger signal (or not, if the trigger signal is a null signal). In some embodiments, the implantable device 508 of the second set 512 detects an additional detection signal (as indicated by arrow 514), and the implantable device 508 analyzes the information related to the detection signal detected by the first set 506 of implantable devices (and optionally the additional information transmitted by the first set 506 of devices) and the detection signal detected by the implantable device 508 of the second set 512 to generate the trigger signal (e.g., update a dynamic state of the device) for the implantable device 508 of the second set 510. Optionally, the implantable device 508 of the second set 512 can generate the trigger signal (e.g., update a dynamic state for the device) based on additional information, such as the location or dynamic state of the implantable device 508, or another implantable device (e.g., 510) in the second set 512. For example, the second implantable device 510 in the second set 512 may wirelessly communicate with the first implantable device 508 in the second set 512 to wirelessly transmit information. The implantable device 508 can then emit an electrical pulse based on the trigger signal, or not (based on the trigger signal). The second implantable device 510 in the second set 512 can operate in a similar manner as the first implantable device 508 in the second set, and can emit an electrical pulse (or not emit an electrical pulse) based on a generated trigger signal (e.g., an updated dynamic state), which was generated on the information received from one or more implantable devices in the first set 506, one or more other devices in the second set 512, and/or a detection signal detected by the second implantable device 510 in the second set 512.

The implantable devices 508 and/or 510 in the second set 512 can wirelessly transmit information back to the implantable devices 502 and/or 504 in the first set 506. The information transmitted by the implantable devices of the second set 512 to the first set 506 of implantable devices can include a detection signal detected by one or more of the implantable devices in the second set 512, information related to an electrical pulse emitted by one or more of the implantable devices in the second set 512, or information related to the dynamic state of one or more of the implantable devices in the second set 512 (for example, a trigger signal generated by one or more of the implantable devices). Once the implantable devices in the first set 506 receive the information from the implantable devices in the second set 512, the dynamic state of the implantable devices 502 and 504 in the first set 506 can be updated based on the received information. The dynamic state of the first implantable device 502 in the first set 506 and the second implantable device 504 in the first set 506 can also or alternatively be updated based on a detection signal detected by the first implantable device 502 or the second implantable device 504 (which may be the same detection signal information transmitted to the second implantable device as previously discussed, or a new detection signal detected by the first or second implantable devices of the first set 506), as indicated by arrow 518 and arrow 520. In some embodiments, the implantable devices within the first set 506 can wirelessly communicate between the devices (e.g., implantable device 502 and implantable device 504 can wirelessly communicate with each other) to transmit additional information, such as detection signal components or a dynamic state of the device. Once the dynamic state of the first implantable device 502 and/or second implantable device 504 in the first set 506 has been updated (i.e., a trigger signal has been generated for the first or second implantable device), the first implantable device 502 and/or second implantable device 504 can emit an electrical pulse to modulate neural activity (or not) based on the trigger signal, if the first implantable device or the second implantable device are configured to do so.

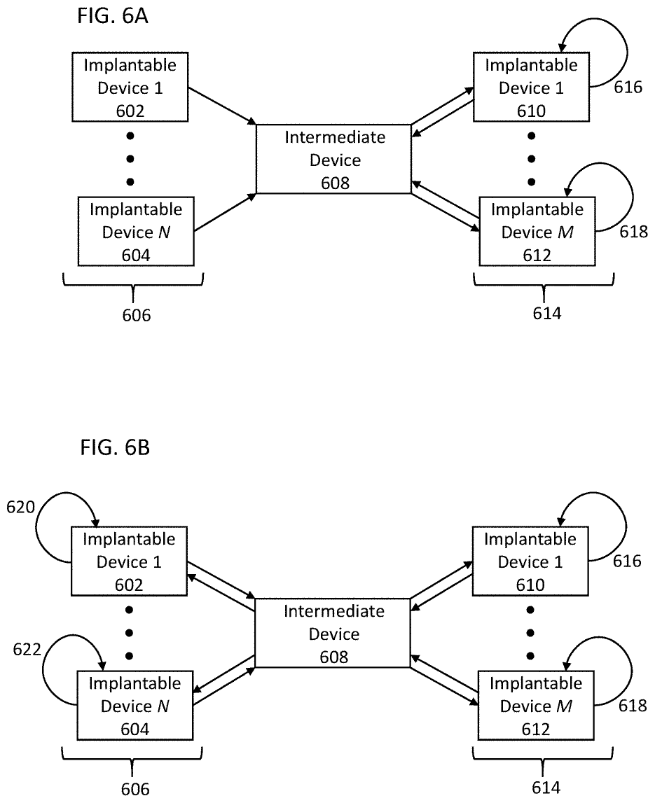

FIG. 6A illustrates a device network with two or more implantable devices (e.g., 602 and 604) in a first set 606 of implantable devices, an intermediate device 608, and two or more implantable devices (e.g., 610 and 612) in a second set 614 of implantable devices. Implantable device 604 is shown as “Implantable Device N” to indicate that any number of implantable devices can be included in the first set 606 of the network, wherein N is 2 or more (e.g., 2, 3, 4, 5, 6, 7, 8, 9, 10, or more). Similarly, implantable device 612 is shown as “Implantable Device M” to indicate that any number of implantable devices can be included in the second set 614 of the network, wherein M is 2 or more (e.g., 2, 3, 4, 5, 6, 7, 8, 9, 10, or more). For simplification, the following describes the network in reference to two implantable devices in the first set 606 and two implantable devices in the second set 614, but is understood that the number of implantable devices in the first set 606 or the second set 614 can be expanded. Additionally, although FIG. 6A illustrates a single intermediate device 608, it is contemplated that one or more (e.g., 2, 3, 4, or more) intermediate devices can be used by the network. That is, a first intermediate device can receive the information related to the detection signal, and the first intermediate device can transmit the information related to the detection signal to a second intermediate device. The second intermediate device can then transmit the information to the two or more implantable devices, or to a third intermediate device.

The first implantable device 602 of the first set 606 detects a first detection signal, and the second implantable device 604 of the first set 606 detects a second detection signal. Information related to the first detection signal and information related to the second detection signal (collectively, the detection signals) are wirelessly transmitted from the two or more implantable devices (602 and 604) of the first set 606, and the information is received by the intermediate device 608. The information transmitted by the implantable devices in the first set 606 may include additional information, such as a dynamic state of one or more of the implantable devices 602 or 604 in the first set 606, a location of one or more of the implantable devices in the first set 606, or information about an electrical pulse emitted by one or more of the implantable devices in the first set 606. The intermediate device 608 then transmits the information related to the detection signal (and, optionally, some or all of the other information transmitted by the implantable devices in the first set 606) from the intermediate device, and the information is wirelessly received by the first implantable device 610 and the second implantable device 612 in the second set 614. The first implantable device 610 can emit one or more electrical pulses configured to modulate neural activity of a first nerve position based on the received information, including the information related to the detection signal, and the second implantable device 612 can emit one or more electrical pulses configured to modulate neural activity of a second nerve position based on the received information, including the information related to the detection signal. In some embodiments, the first implantable device 610 and/or the second implantable device 610 in the second set 614 can detect an additional detection signal, which may be included in the information used as a basis for generating the electrical pulse(s).

In some embodiments, the intermediate device 608 functions as a relay, and receives the information from the two or more implantable devices (602 and 604) in the first set 606, and transmits the information to the second set 614 of implantable devices without analyzing the information. The two or more implantable devices (610 and 612) in the second set receive the information related to, including the information related to the detection signal detected by the first set 606, and the implantable devices 610 and 612 analyze the information to generate a trigger signals (e.g., updates a dynamic status) for the respective devices. The first implantable device 610 of the second set can then emit (or not emit, if the trigger signal is a null signal) one or more electrical pulses based on the trigger signal (e.g., dynamic status) for the first implantable device 610, and the second implantable device 612 of the second set 614 can emit one or more electrical pulses based on a trigger signal for the second implantable device 612 of the second set 614. Optionally, the first implantable device 610 of the second set 614 detects an additional detection signal, and the first implantable device 610 analyzes the information from the first set 606 of implantable devices and the detection signal detected by the first implantable device 610 of the second set 614 (and, optionally, additional information from the implantable device 610 in the second set 614, such as a dynamic state or a location of the device) to generate the trigger signal of the first implantable device 610 of the second set 614. Optionally or additionally, the second implantable device 612 of the second set 614 detects an additional detection signal, and the second implantable device 612 analyzes the information from the first set 606 of implantable devices and the detection signal detected by the second implantable device 612 of the second set 614 (and, optionally, additional information from the implantable device 612 in the second set 614, such as a dynamic state or a location of the device) to generate the trigger signal for the second implantable device 612 of the second set 614.

In some embodiments, the intermediate device 608 analyzes the information received from the first set 606 of implantable devices to generate a trigger signal for one or more of the implantable devices in the second set 614. Optionally, the two or more implantable devices (608 and 610) detect one or more additional detection signals, which are wirelessly transmitted to the intermediate device 608. The implantable devices in the second set 614 can wirelessly transmit additional information to the intermediate device 608, such as a dynamic state of one or more of the implantable devices in the second set 614, or a location or unique identification of the one or more implantable devices in the second set 614. The intermediate device can analyze the information (including information related to a detection signal) from the two or more implantable devices (602 and 604) in the first set 606, and the information from the implantable devices in the second set 614 to generate the trigger signals for the two or more implantable devices in the second set 614. One or more of the two or more implantable devices in the second set 614 can then emit (or not emit) one or more electrical pulses configured to modulate neural activity of a nerve positions based on the one or more trigger signals.

FIG. 6B shows a device network configured according to a recurrent neural network, with includes a first set of implantable devices and a second set of implantable devices, which are configured to wirelessly communicate through an intermediate device using bidirectional communication. The implantable devices 602 and 604 in the first set 606 wirelessly transmit information related to a detection signal to an intermediate device 608. The information transmitted by the implantable devices in the first set 606 may include additional information, such as a dynamic state of one or more of the implantable devices 602 or 604 in the first set 406, a location of one or more of the implantable devices in the first set 606, or information about an electrical pulse emitted by one or more of the implantable devices in the first set 606. The intermediate device 608 then wirelessly transmits the information related to the detection signal (and, optionally, other information transmitted by the implantable devices in the first set 606) to the implantable devices 610 and 612 in the second set 614, which can emit one or more electrical pulses that modulate neural activity of a target nerve based on the received information.

The intermediate device 608 can wirelessly relay the information received from the two or more implantable devices (602 and 604) in the first set 606 to the one or more implantable devices 610 or 612 in the second set 614 by receiving the information from the first set 606 of implantable devices and wirelessly transmitting the information to the implantable devices 610 and 612 in the second set 412, either with or without analyzing the information. The implantable devices 610 and 612 in the second set 614 then receive the information, including the information related to the detection signal detected by the implantable devices in the first set 606 (and optionally the additional information transmitted by the implantable devices in the first set 606), and analyze the received information to generate a trigger signal for respective device (e.g., update a dynamic state of the first implantable device 610 and update a dynamic state of the second implantable device 612), if the information was not pre-analyzed by the intermediate device 608. Optionally, the implantable device 610 detects an additional detection signal, and the implantable device 610 analyzes the information related to the detection signal detected by the two or more implantable devices (602 and 604) in the first set 606 (and, optionally, some or all of the additional information transmitted by the first set 606 of implantable devices) and the detection signal detected by the first implantable device 610 in the second set 612 to generate the trigger signal (e.g., update the dynamic state) of the implantable device 610, as indicated by arrow 616. Once the implantable device 610 generates the trigger signal, it can emit one or more electrical pulses that modulate neural activity of a target nerve (or not emit such a pulse) based on the trigger signal. The second implantable device 612 in the second set 614 (and any other device in the second set) can optionally operate in a similar manner as the first implantable device 610 in the second set 614. The first implantable device 610 and the second implantable device 612 in the second set 614 may also wirelessly communicate information to each other, either directly or through an intermediate device, and this information can be used as a basis for updating the dynamic state of the device.

The implantable devices 610 and/or 612 in the second set 614 can wirelessly transmit information back to the intermediate device 608, which can wirelessly transmit the information (either with or without analysis) to other implantable devices in the second set 614 or to the one or more implantable devices in the first set 606. The information transmitted by the implantable devices of the second set 614 to the intermediate device 608 can include a detection signal detected by one or more of the implantable devices in the second set 614, information related to an electrical pulse emitted by one or more of the implantable devices in the second set 614, or information related to the dynamic state of one or more of the implantable devices in the second set 614 (for example, a trigger signal generated by one or more of the implantable devices). Once the implantable devices in the first set 606 receive the information from the implantable devices in the second set 614, the dynamic state of the implantable devices 602 and 604 in the first set 606 can be updated based on the received information. The dynamic state of the first implantable device 602 in the first set 606 and the second implantable device 604 in the first set 606 can also or alternatively be updated based on a detection signal detected by the first implantable device 602 or the second implantable device 604 (which may be the same detection signal information transmitted to the second implantable device as previously discussed, or a new detection signal detected by the first or second implantable devices of the first set 606), as indicated by arrow 620 and arrow 622. In some embodiments, the implantable devices within the first set 606 can wirelessly communicate between the devices (e.g., implantable device 602 and implantable device 606 can wirelessly communicate with each other) directly or through the intermediate device 608 to transmit additional information, such as detection signal components or a dynamic state of the device. Once the dynamic state of the first implantable device 602 and/or second implantable device 604 in the first set 606 has been updated (i.e., a trigger signal has been generated for the first or second implantable device), the first implantable device 602 and/or second implantable device 604 can emit an electrical pulse to modulate neural activity (or not) based on the trigger signal, if the first implantable device or the second implantable device are configured to do so.

### Implantable Devices

The implantable device networks described herein include two or more implantable devices. Some or all of the devices within the network can be configured to both detect a detection signal and emit an electrical pulse configured to modulate neural activity. In some embodiments of the network, a portion of the implantable devices may be configured to detect a detection signal and not emit an electrical pulse, while another portion of the devices may be configured to emit an electrical pulse but not detect a detection signal.

Generally, at least one implantable device is configured to detect a detection signal and wirelessly transmit information related to the detection signal. At least one other implantable device is configured to receive the information related to the detection signal (which is optionally analyzed prior to receipt by the implantable device such as by the implantable device or by an intermediate device), and emit one or more electrical pulses configured to modulate neural activity of a nerve based on the received information related to the detection signal.

In some embodiments, the one or more implantable devices wirelessly communicate with another device using ultrasonic waves. The ultrasonic waves received by the device may encode information, which can be decoded by the device. In some embodiments, one or more implantable devices backscatters ultrasonic waves, and encode information in the ultrasonic backscatter waves to wirelessly transmit the information. In some embodiments, the one or more implantable devices actively transmit ultrasonic waves that encode information. The information wirelessly transmitted by the implantable devices (whether by ultrasonic waves generated and actively transmitted by the device or ultrasonic waves backscattered by the device) can be received by a separate device, which may be another implantable device, or an intermediate device. If the implantable device communicates using ultrasonic waves, the implantable device includes one or more ultrasonic transducers, which receive, actively transmit, or backscatter the ultrasonic waves. Additionally or alternatively, the implantable device can include one or more ultrasonic transducers configured to receive ultrasonic waves that power the device. Ultrasonic transducers configured to wirelessly communicate and ultrasonic transducers configured to receive ultrasonic waves that power the implantable device may be the same or different ultrasonic transducers.

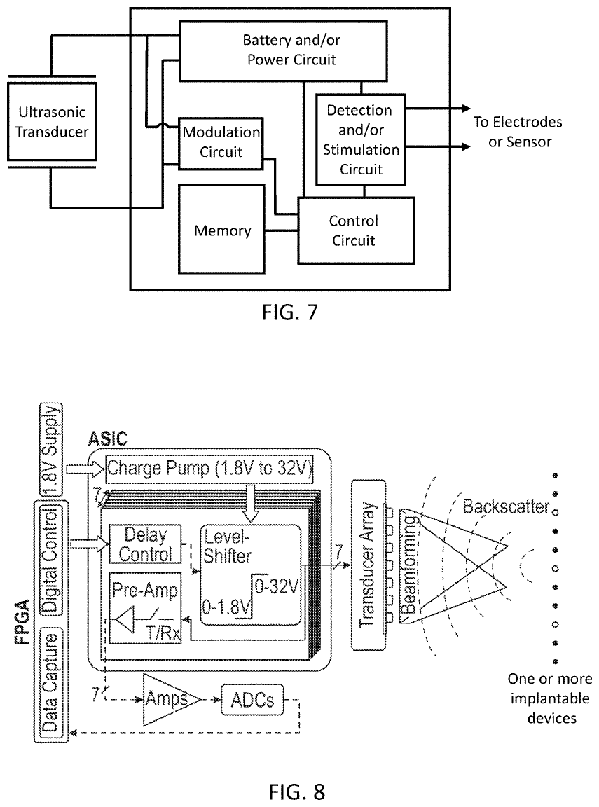

FIG. 7 illustrates a schematic of an exemplary body for the implantable device described herein. The implantable device may further include electrodes extending from the device, which can be positioned in electrical communication with a nerve to emit an electrical pulse to modulate activity of the nerve or detect an electrophysiological signal transmitted by the nerve. The body includes an ultrasonic transducer electrically connected to a battery and/or power circuit, and a modulation circuit. The battery and/or power circuit is electrically connected to and powers a control circuit, which is electrically connected to an optional non-transitory memory and the modulation circuit. The control circuit is also electrically connected and is configured to operate a stimulation circuit and/or a detection circuit. Ultrasonic waves are received by the ultrasonic transducer, which converts the energy from the ultrasonic waves into an electrical energy that charges the battery or one or more capacitors on the power circuit. Electrodes on the device may be configured to detect an electrophysiological signal or a physiological condition, and a detection signal based on the electrophysiological signal or physiological condition is received by the control circuit. The detection signal received by the control circuit may be processed (for example, amplified, digitized, and/or filtered) by the detection circuit before being received by the computational circuit. Optionally, the control circuit accesses the non-transitory memory to store data related to the detection signal. If the implantable device is configured to emit an electrical pulse, the control circuit can generate a stimulation signal, and operate the electrodes from a stimulation circuit to emit an electrical pulse to the nerve based on the stimulation signal. Optionally, the computational circuit accesses the non-transitory memory to store data related to the stimulation signal or electrical pulse emitted to the nerve. Data stored on the non-transitory memory can be wirelessly transmitted through ultrasonic backscatter waves or ultrasonic waves generated by the ultrasonic transducer. The control circuit accesses the memory and operates the modulation circuit to modulate the current flowing through the modulation circuit to encode the data on the ultrasonic waves.

In some embodiments, the body includes a housing, which can include a base, one or more sidewalls, and a top. The housing can enclose the one or more ultrasonic transducers and the integrated circuit (which includes the computational circuit, the non-transitory memory, the battery, the modulation circuit, a detection circuit, and/or a stimulation circuit). The housing may be sealed closed (for example by soldering or laser welding) to prevent interstitial fluid from coming in contact with the ultrasonic transducer(s) and/or the integrated circuit. The housing is preferably made from a bioinert material, such as a bioinert metal (e.g., steel or titanium) or a bioinert ceramic (e.g., titania or alumina). The housing (or the top of the housing) may be thin to allow ultrasonic waves to penetrate through the housing. In some embodiments, the thickness of the housing is about 100 micormeters (μm) or less in thickness, such as about 75 μm or less, about 50 μm or less, about 25 μm or less, or about 10 μm or less. In some embodiments, the thickness of the housing is about 5 μm to about 10 μm, about 10 μm to about 25 μm, about 25 μm to about 50 μm, about 50 μm to about 75 μm, or about 75 μm to about 100 μm in thickness.

The body of the implantable device is relatively small, which allows for comfortable and long-term implantation while limiting tissue inflammation that is often associated with implantable devices. In some embodiments, the longest dimension of the body of the device is about 10 mm or less, such as about 5 mm to about 9 mm, or about 6 mm to about 8 mm.

In some embodiments, the body comprises a material, such as a polymer, within the housing. The material can fill empty space within the housing to reduce acoustic impedance mismatch between the tissue outside of the housing and within the housing. Accordingly, the body of the device is preferably void of air or vacuum.

The ultrasonic transducer of the implantable device can be a micro-machined ultrasonic transducer, such as a capacitive micro-machined ultrasonic transducer (CMUT) or a piezoelectric micro-machined ultrasonic transducer (PMUT), or can be a bulk piezoelectric transducer. Bulk piezoelectric transducers can be any natural or synthetic material, such as a crystal, ceramic, or polymer. Exemplary bulk piezoelectric transducer materials include barium titanate (BaTiO₃), lead zirconate titanate (PZT), zinc oxide (ZO), aluminum nitride (AlN), quartz, berlinite (AlPO₄), topaz, langasite (La₃GasSiO₁₄), gallium orthophosphate (GaPO₄), lithium niobate (LiNbO₃), lithium tantalite (LiTaO₃), potassium niobate (KNbO₃), sodium tungstate (Na₂WO₃), bismuth ferrite (BiFeO₃), polyvinylidene (di)fluoride (PVDF), and lead magnesium niobate-lead titanate (PMN-PT).

In some embodiments, the bulk piezoelectric transducer is approximately cubic (i.e., an aspect ratio of about 1:1:1 (length:width:height). In some embodiments, the piezoelectric transducer is plate-like, with an aspect ratio of about 5:5:1 or greater in either the length or width aspect, such as about 7:5:1 or greater, or about 10:10:1 or greater. In some embodiments, the bulk piezoelectric transducer is long and narrow, with an aspect ratio of about 3:1:1 or greater, and where the longest dimension is aligned to the direction of the ultrasonic backscatter waves (i.e., the polarization axis). In some embodiments, one dimension of the bulk piezoelectric transducer is equal to one half of the wavelength (λ) corresponding to the drive frequency or resonant frequency of the transducer. At the resonant frequency, the ultrasound wave impinging on either the face of the transducer will undergo a 180° phase shift to reach the opposite phase, causing the largest displacement between the two faces. In some embodiments, the height of the piezoelectric transducer is about 10 μm to about 1000 μm (such as about 40 μm to about 400 μm, about 100 μm to about 250 μm, about 250 μm to about 500 μm, or about 500 μm to about 1000 μm). In some embodiments, the height of the piezoelectric transducer is about 5 mm or less (such as about 4 mm or less, about 3 mm or less, about 2 mm or less, about 1 mm or less, about 500 μm or less, about 400 μm or less, 250 μm or less, about 100 μm or less, or about 40 μm or less). In some embodiments, the height of the piezoelectric transducer is about 20 μm or more (such as about 40 μm or more, about 100 μm or more, about 250 μm or more, about 400 μm or more, about 500 μm or more, about 1 mm or more, about 2 mm or more, about 3 mm or more, or about 4 mm or more) in length.

In some embodiments, the ultrasonic transducer has a length of about 5 mm or less such as about 4 mm or less, about 3 mm or less, about 2 mm or less, about 1 mm or less, about 500 μm or less, about 400 μm or less, 250 μm or less, about 100 μm or less, or about 40 μm or less) in the longest dimension. In some embodiments, the ultrasonic transducer has a length of about 20 μm or more (such as about 40 μm or more, about 100 μm or more, about 250 μm or more, about 400 μm or more, about 500 μm or more, about 1 mm or more, about 2 mm or more, about 3 mm or more, or about 4 mm or more) in the longest dimension.

The ultrasonic transducer is connected to two electrodes to allow electrical communication with an integrated circuit. The first electrode is attached to a first face of the transducer and the second electrode is attached to a second face of the transducer, wherein the first face and the second face are opposite sides of the transducer along one dimension. In some embodiments, the electrodes comprise silver, gold, platinum, platinum-black, poly(3,4-ethylenedioxythiophene (PEDOT), a conductive polymer (such as conductive PDMS or polyimide), or nickel. In some embodiments, the axis between the electrodes of the transducer is orthogonal to the motion of the transducer.

The integrated circuit of the implantable device can include a control circuit (e.g., a digital circuit, a mixed-signal integrated circuit, or a computational circuit) and a modulation circuit, which can be operated by the control circuit. The modulation circuit is electrically connected to the one or more ultrasonic transducers and can modulate ultrasonic waves, which may be ultrasonic backscatter waves or actively generated (i.e., transduced) ultrasonic waves, to encode data. The integrated circuit of a device can include a detection circuit coupled to a sensor configured to detect a detection signal, and the detection circuit can be operated by the control circuit. The integrated circuit of an implantable device can also or alternatively include a stimulation circuit coupled to a plurality of electrodes. The stimulation circuit is configured to emit an electrical pulse to a target nerve to modulate neural activity of the nerve, and can be operated by the control circuit.

The control circuit may include a memory and one or more circuit blocks, systems, or processors for operating the implantable device. These systems can include, for example, an onboard microcontroller or processor, a finite state machine (FSM), a field programmable gate array (FPGQ), or digital circuits capable of executing one or more programs stored on the implantable device. In some embodiments, the control circuit includes an analog-to-digital converter (ADC), which can convert analog signal encoded in the ultrasonic waves emitted from a separate device so that the signal can be processed by the control circuit. In some embodiments, the integrated circuit includes a volatile memory, which can be accessed by the computational circuit. As further described herein, a computational circuit of an implantable device may be used to analyze the detection signal to generate a trigger signal.

The modulation circuit of the implantable device modulates an electrical current flowing through the one or more ultrasonic transducers to encode data in the electrical current. The modulation circuit includes one or more switches, such as an on/off switch or a field-effect transistor (FET). An exemplary FET that can be used with some embodiments of the implantable device is a metal-oxide-semiconductor field-effect transistor (MOSFET). The modulation circuit can alter the impedance of a current flowing through the ultrasonic transducer, and variation in current flowing through the transducer encodes the information. Alternatively, if the information is transmitted through actively transmitted ultrasonic waves, the integrated circuit can operate the ultrasonic transducer to actively transmit ultrasonic waves encoding the information. In some embodiments, the modulation circuit is operated by a control circuit (e.g., a computational circuit, a digital circuit, or a mixed-signal integrated circuit), which can actively encode the information in a digitized or analog signal.

The integrated circuit can further include a power circuit, which can include an energy storage circuit. The implantable device powered by ultrasonic waves may be batteryless, although the energy storage circuit can include one or more capacitors to temporarily store electrical energy. However, in some embodiments, the implantable device comprises a battery configured to store energy that powers the device. Energy from the ultrasonic waves can be converted into a current by the ultrasonic transducer, and can be stored in the energy storage circuit, which can include one or more capacitors. The energy can be used to operate the implantable device, such as providing power to the digital circuit, the modulation circuit, or one or more amplifiers, or can be used to generate the electrical pulse used to stimulate the tissue. In some embodiments, the power circuit further includes, for example, a rectifier and/or a charge pump.

In some embodiments, the implantable device further includes a battery configured to receive the electrical energy from the one or more ultrasonic transducers and power the computational circuit. Inclusion of the battery allows the computational circuit to function without an external power source, including detecting an electrophysiological signal or emitting an electrical pulse to the nerve. The battery can be contained within the body of the implantable device. The battery can be, for example, a rechargeable electrochemical battery. The energy stored by the battery can power the device, for example when the one or more ultrasonic transducers are not receiving ultrasonic waves. The battery can be charged by transmitting ultrasonic waves to the device using a separate device, which are received by the one or more ultrasonic transducers. The one or more ultrasonic transducers convert the ultrasonic waves into an electrical energy, and are electrically connected to the battery. In this manner, the electrical energy charges the battery of the device.

The implantable device can also include a non-transitory memory configured to store data based on a detection signal detected by the device or information related to an electrical pulse emitted by the device. The data can include, for example, a time stamp, a velocity, a direction, an amplitude, a frequency, or a waveform of a detected action potential or compound action potential; and/or a time stamp, an amplitude, a frequency, or a waveform of an electrical pulse emitted by implantable device. In some embodiments, the non-transitory memory can store data related to a detected physiological condition (such as temperature, pH, pressure, heart rate, strain, and/or presence or amount of an analyte). The data stored on the non-transitory memory may be acquired over a period of time (such as about 1 minute or more, about 5 minutes or more, about 10 minutes or more, about 15 minutes or more, about 30 minutes or more, about 45 minutes or more, about 1 hour or more, about 2 hours or more, about 4 hours our more, about 6 hours or more, about 8 hours or more, about 12 hours or more, or about 24 hours or more).

The non-transitory memory can also be used to store data transmitted to the device from a separate device, such as a separate implantable device or an intermediate device. The separate device can transmit data (such a detection signal or a trigger signal), which is received by the implantable device and can be stored on the non-transitory memory. The data can be transmitted, for example, through ultrasonic waves that encode the data. The separate device can transmit the ultrasonic waves, which are received by the ultrasonic transducer of the device and deciphered by the computational circuit.

Optionally, the non-transitory memory stores one or more instructions for operating the device, which can be executed using the control circuit. For example, the non-transitory memory can include instructions for receiving a detection signal based: generating a trigger signal; generating or retrieving a stimulation signal, and/or operating a sensor on the implantable device. In some embodiments, the non-transitory memory includes instructions for selectively activating one or more electrodes with the plurality of electrodes for targeted emission of the electrical pulse.

At least a portion of the implantable devices of the networks described herein are configured to detect a detection signal and wirelessly transmit from the implantable device information related to the detection signal. Such an implantable device includes one or more ultrasonic transducers configured to actively transmit ultrasonic waves that encode the information related to the detection signal, or backscatter ultrasonic waves that encode the information related to the detection signal. The implantable device further includes one or more sensors configured to detect the detection signal. The one or more sensors can be configured to detect a physiological condition or an electrophysiological signal transmitted by a nerve, or both. In some embodiments, the detection signal includes two or more components, and the implantable device can include two or more different sensors to detect different components of the detection signal. For example, an implantable device can include a first sensor comprising a plurality of electrodes configured to detect an electrophysiological signal and a second sensor configured to detect a physiological condition (such as a pH, temperature, amount of an analyte (e.g., concentration), presence of an analyte, pressure, bioimpedence, or strain). Exemplary implantable devices are described in US 2018/0085605; WO 2018/009905 WO 2018/009910, and WO 2018/009911. The analyte detected (either amount or presence) by the implantable device can be, for example, glucose or oxygen.

The one or more sensors of the implantable device can be coupled to a detection circuit, which is operated by a control circuit. The control circuit can store information related to the detection signal on a memory and/or operate a modulation circuit to encode the information in ultrasonic waves generated by or backscattered from the ultrasonic transducer. In some embodiments, the control circuit analyzes the detection signal to generate a trigger signal, which can be encoded in the ultrasonic waves generated by or backscattered from the device.

In some embodiments, the implantable device comprises a sensor configured to detect a physiological condition, such as temperature, pH, an analyte amount (such as glucose or oxygen), the presence of an analyte, a strain, or a pressure. In some embodiments, the implantable device includes a sensor configured to detect an electrophysiological signal, which can include a plurality of electrodes in electrical communication with a nerve. The implantable device may comprise one or more (such as 2, 3, 4, 5 or more) sensors, which may detect the same physiological condition or different physiological conditions. In some embodiments, the implantable device comprises 10, 9, 8, 7, 6 or 5 or fewer sensors). For example, in some embodiments, the implantable device comprises a first sensor configured to detect temperature and a second sensor configured to detect oxygen. Changes in both physiological conditions can be encoded in the ultrasonic backscatter waves, which can be deciphered by an external computing system.

Additionally, one or more implantable devices of the network can emit one or more electrical pulses that modulate neural activity of a target nerve based on the information related to the detection signal received by the implantable device. In some embodiments, the implantable device configured to emit an electrical pulse can also detect an additional detection signal (e.g., a physiological condition and/or an electrophysiological signal), and the implantable device emits the one or more electrical pulses based on the information related to the detection signal detected by a different implantable device and the information related to the additional detection signal.

The implantable device configured to emit the electrical pulse includes a plurality of electrodes electrically coupled to the stimulation circuit of the implantable device. The electrodes are positioned in electrical communication with a target nerve, and can emit an electrical pulse that modulated neural activity of the target nerve. In some embodiments, an electrical pulse emitted by the implantable device stimulates an action potential in the tissue. In some embodiments, an electrical pulse emitted by the implantable device blocks an action potential in a tissue. The control circuit of the implantable device generates a stimulation signal based on a trigger signal, and operates the stimulation circuit using the stimulation signal. For example, the stimulation signal can include a pulse amplitude, frequency, and/or waveform, and the control circuit controls the electrodes via the stimulation circuit to emit the pulse in accordance with the stimulation signal.

The stimulation circuit of the implantable device configured to emit an electrical pulse can include a stimulating capacitor, which can be charged by the battery, power circuit, or electrical energy converted from the ultrasonic waves by the one or more ultrasonic transducers. The status of the stimulating capacitor, for example capacitor charge, can be determined by the computational circuit. Optionally, the status of the stimulating capacitor is recorded on the non-transitory memory or encoded in ultrasonic backscatter waves through the modulation circuit operated by the computational circuit. In some embodiments, the control circuit is configured to determine a stimulating capacitor status, such as a charge of the capacitor. The capacitor status can be stored in the non-transitory memory and/or encoded in ultrasonic backscatter waves.

In some embodiments, the control circuit of the implantable device analyzes the information related to the detection signal and generates a trigger signal. The trigger signal is then used by the control circuit to generate a stimulation signal, and the control circuit operates the stimulation circuit to emit the one or more electrical pulses. In some embodiments, the control circuit extracts the trigger signal from data encoded in ultrasonic waves received by the implantable device (e.g., ultrasonic waves backscattered or generated by a different implantable device or an intermediate device). The control circuit can then generate the stimulation signal based on the trigger signal, and operate the stimulation circuit to emit one or more electrical pulses based on the stimulation signal.

### Intermediate Devices

The networks described herein can optionally include one or more intermediate devices, which can be used to connect two or more implantable devices tin the network. Although the network can include direct wireless communication between the implantable devices, the intermediate device can be used to relay a communication (e.g., the data based on the detection signal) to one or more implantable devices or can process information, such as information related to a detection signal, to generate a trigger signal, which is wirelessly communicated to one or more implantable devices. In some embodiments, the intermediate device can also wirelessly power one or more of the implantable devices in the network through ultrasonic waves.

The intermediate device is configured to receive ultrasonic waves that encode information, and generate ultrasonic waves that encode information. For example, the intermediate device can generate ultrasonic waves, which are received by a first device. The first device backscatters the ultrasonic waves and encodes information in the ultrasonic backscatter, which is received by the intermediate device. The intermediate device can then extract the information, and optionally analyze the information. The intermediate device can then generate ultrasonic waves that encode the information or the analyzed information (e.g., a trigger signal), which are received by a second implantable device. The second implantable device can then extract the information from the ultrasonic waves. The intermediate device can receive information from a plurality of devices, and relay or analyze the information from the plurality of devices.

The intermediate device includes one or more ultrasonic transducers, which can operate as an ultrasonic transmitter and/or an ultrasonic receiver (or as a transceiver, which can be configured to alternatively transmit or receive the ultrasonic waves). The one or more transducers can be arranged as a transducer array, and the intermediate device can optionally include one or more transducer arrays. In some embodiments, the ultrasound transmitting function is separated from the ultrasound receiving function on separate devices. That is, optionally, the intermediate device comprises a first component that transmits ultrasonic waves to one or more implantable devices, and a second component that receives ultrasonic waves from one or more implantable device. In some embodiments, the transducers in the array can have regular spacing, irregular spacing, or be sparsely placed. In some embodiments the array is flexible. In some embodiments the array is planar, and in some embodiments the array is non-planar.

A schematic of an exemplary intermediate device is shown in FIG. 8. The illustrated intermediate device shows a transducer array with a plurality of ultrasonic transducers. In some embodiments, the transducer array includes 1 or more, 2 or more, 3 or more, 5 or more, 7 or more, 10 or more, 15 or more, 20 or more, 25 or more, 50 or more, 100 or more 250 or more, 500 or more, 1000 or more, 2500 or more, 5000 or more, or 10,000 or more transducers. In some embodiments, the transducer array includes 100,000 or fewer, 50,000 or fewer, 25,000 or fewer, 10,000 or fewer, 5000 or fewer, 2500 or fewer, 1000 or fewer, 500 or fewer, 200 or fewer, 150 or fewer, 100 or fewer, 90 or fewer, 80 or fewer, 70 or fewer, 60 or fewer, 50 or fewer, 40 or fewer, 30 or fewer, 25 or fewer, 20 or fewer, 15 or fewer, 10 or fewer, 7 or fewer or 5 or fewer transducers. The transducer array can be, for example a chip comprising 50 or more ultrasonic transducer pixels.

The intermediate device shown in FIG. 8 illustrates a single transducer array; however the intermediate device can include 1 or more, 2 or more, or 3 or more separate arrays. In some embodiments, the intermediate device includes 10 or fewer transducer arrays (such as 9, 8, 7, 6, 5, 4, 3, 2, or 1 transducer arrays). The separate arrays, for example, can be placed at different points of a subject, and can communicate to the same or different implantable devices. In some embodiments, the arrays are located on opposite sides of an implantable device. The intermediate can include an application specific integrated circuit (ASIC), which includes a channel for each transducer in the transducer array. In some embodiments, the channel includes a switch (indicated in FIG. 8 by “T/Rx”). The switch can alternatively configure the transducer connected to the channel to transmit ultrasonic waves or receive ultrasonic waves. The switch can isolate the ultrasound receiving circuit from the higher voltage ultrasound transmitting circuit.

In some embodiments, the transducer connected to the channel is configured only to receive or only to transmit ultrasonic waves, and the switch is optionally omitted from the channel. The channel can include a delay control, which operates to control the transmitted ultrasonic waves. The delay control can control, for example, the phase shift, time delay, pulse frequency and/or wave shape (including amplitude and wavelength). The delay control can be connected to a level shifter, which shifts input pulses from the delay control to a higher voltage used by the transducer to transmit the ultrasonic waves. In some embodiments, the data representing the wave shape and frequency for each channel can be stored in a ‘wave table’. This allows the transmit waveform on each channel to be different. Then, delay control and level shifters can be used to ‘stream’ out this data to the actual transmit signals to the transducer array. In some embodiments, the transmit waveform for each channel can be produced directly by a high-speed serial output of a microcontroller or other digital system and sent to the transducer element through a level shifter or high-voltage amplifier. In some embodiments, the ASIC includes a charge pump (illustrated in FIG. 8) to convert a first voltage supplied to the ASIC to a higher second voltage, which is applied to the channel. The channels can be controlled by a controller, such as a digital controller, which operates the delay control.

In the ultrasound receiving circuit, the received ultrasonic waves are converted to current by the transducers (set in a receiving mode), which is transmitted to a data capture circuit. In some embodiments, an amplifier, an analog-to-digital converter (ADC), a variable-gain-amplifier, or a time-gain-controlled variable-gain-amplifier which compensates for tissue loss, and/or a band pass filter is included in the receiving circuit. The ASIC can draw power from a power supply, such as a battery (which is preferred for a wearable embodiment of the intermediate device). In the embodiment illustrated in FIG. 8, a 1.8V supply is provided to the ASIC, which is increased by the charge pump to 32V, although any suitable voltage can be used. In some embodiments, the intermediate device includes a processor and or a non-transitory computer readable memory. In some embodiments, the channel described above does not include a T/Rx switch but instead contains independent Tx (transmit) and Rx (receive) with a high-voltage Rx (receiver circuit) in the form of a low noise amplifier with good saturation recovery. In some embodiments, the T/Rx circuit includes a circulator. In some embodiments, the transducer array contains more transducer elements than processing channels in the device transmit/receive circuitry, with a multiplexer choosing different sets of transmitting elements for each pulse. For example, 64 transmit receive channels connected via a 3:1 multiplexer to 192 physical transducer elements—with only 64 transducer elements active on a given pulse.

In some embodiments, the intermediate device includes a computational circuit configured to analyze the information related to the detection signal to generate a trigger signal. The trigger signal can then be wirelessly communicated to one or more implantable device, which controls the implantable devices to emit an electrical pulse in accordance with the trigger signal.

In some embodiments, the intermediate device is implantable. In some embodiments, the intermediate device is external (i.e., not implanted). By way of example, the external intermediate device can be a wearable, which may be fixed to the body by a strap or adhesive. In another example, the external intermediate device can be a wand, which may be held by a user (such as a healthcare professional). In some embodiments, the intermediate device can be held to the body via suture, simple surface tension, a clothing-based fixation device such as a cloth wrap, a sleeve, an elastic band, or by sub-cutaneous fixation. The transducer or transducer array of the intermediate device may be positioned separately from the rest of the transducer. For example, the transducer array can be fixed to the skin of a subject at a first location (such as proximal to one or more implanted devices), and the rest of the intermediate device may be located at a second location, with a wire tethering the transducer or transducer array to the rest of the intermediate device.

The specific design of the transducer array depends on the desired penetration depth, aperture size, and size of the individual transducers within the array. The Rayleigh distance, R, of the transducer array is computed as:

$$R = \frac{{D}^{2} - {λ}^{2}}{4 λ} \approx \frac{{D}^{2}}{4 λ} , {D}^{2} ⪢ {λ}^{2}$$

where D is the size of the aperture and X is the wavelength of ultrasound in the propagation medium (i.e., the tissue). As understood in the art, the Rayleigh distance is the distance at which the beam radiated by the array is fully formed. That is, the pressure filed converges to a natural focus at the Rayleigh distance in order to maximize the received power. Therefore, in some embodiments, the implantable device is approximately the same distance from the transducer array as the Rayleigh distance.

The individual transducers in a transducer array can be modulated to control the Raleigh distance and the position of the beam of ultrasonic waves emitted by the transducer array through a process of beamforming or beam steering. Techniques such as linearly constrained minimum variance (LCMV) beamforming can be used to communicate a plurality of implantable devices with an external ultrasonic transceiver. See, for example, Bertrand et al., Beamforming Approaches for Untethered, Ultrasonic Neural Dust Motes for Cortical Recording: a Simulation Study, IEEE EMBC (Aug. 2014). In some embodiments, beam steering is performed by adjusting the power or phase of the ultrasonic waves emitted by the transducers in an array.

In some embodiments, the intermediate device includes one or more of instructions for beam steering ultrasonic waves using one or more transducers, instructions for determining the relative location of one or more implantable devices, instructions for monitoring the relative movement of one or more implantable devices, instructions for recording the relative movement of one or more implantable devices, and instructions for deconvoluting backscatter from a plurality of implantable devices.

Optionally, the intermediate device is controlled using a separate computer system, such as a mobile device (e.g., a smartphone or a table). The computer system can wirelessly communicate to the intermediate device, for example through a network connection, a radiofrequency (RF) connection, or Bluetooth. The computer system may, for example, turn on or off the intermediate device or analyze information encoded in ultrasonic waves received by the intermediate device.

### Wireless Communication

Information can be wirelessly transmitted from the implantable devices by encoding the information in ultrasonic waves, which may be actively transmitted or backscattered from the implantable devices in the network. The use of ultrasonic waves for wireless communication is preferred over other wireless communication modalities, such as radiofrequency waves, because the ultrasonic waves can efficiently communicate using lower energy than radiofrequency.

The implantable devices in the neuromodulation network can be configured for bidirectional wireless communication. That is, the devices can be configured to wirelessly receive information from one or more separate implantable device and/or one or more intermediate device, and wirelessly transmit information from one or more separate implantable devices and/or one or more intermediate devices. By way of example, an implantable device can be configured to wirelessly receive information related to the detection signal from an intermediate device and/or wirelessly transmit information related to the detection signal to the intermediate device or one or more implantable devices. An implantable device can be configured to wirelessly transmit information to the intermediate device, such as information related to the status of the device or information related to the electrical pulse emitted by the device. In some embodiments, the implantable device is configured to only transmit information or only receive information. The intermediate device(s) are configured for bidirectional wireless communication, and can receive information from an implantable device or other intermediate device, and can transmit information to an implantable device or other intermediate device. In some embodiments, the intermediate device is further configured to wirelessly receive or transmit information from implantable devices.

The implantable devices and/or intermediate devices can include one or more ultrasonic transducers, which can be used for bidirectional or unidirectional (either receiving or transmitting) information. In some embodiments, the device includes a first ultrasonic transducer configured to transmit information, and a second ultrasonic transducer configured to receive information. In some embodiments, the ultrasonic transducer is controlled by a switch that can to selectively configure the ultrasonic transducer in a transmit mode or a receive mode.

The one or more ultrasonic transducers are operated by a computational circuit to encode information (for example, detection signal information) in the ultrasonic waves. In some embodiments, the computational circuit operates the one or more ultrasonic transducers to actively generate ultrasonic waves that encode information.

In some embodiments, the implantable device wirelessly transmits information through ultrasonic backscatter waves. The implantable device receives ultrasonic waves from an intermediate device or other implantable device through the one or more ultrasonic transducers on the implantable device. Vibrations of the ultrasonic transducer(s) on the implantable device generate a voltage across the electric terminals of the transducer, which causes a current to flow through the ultrasonic transducer and a modulation circuit. Ultrasonic backscatter waves are backscattered from the ultrasonic transducer, which can encode information wirelessly transmitted from the implantable device. The information can be encoded, for example, by changes in amplitude, frequency, or phase of the backscattered ultrasound waves. To encode signals in the ultrasonic backscatter waves, current flowing through the ultrasonic transducer(s) of the implantable device is modulated as a function of the encoded information. In some embodiments, modulation of the current can be an analog signal. In some embodiments, modulation of the current encodes a digitized signal, which may be controlled by the computational circuit of the implantable device. The ultrasonic backscatter waves encoding the information are received by the receiving device (i.e., an implantable device or an intermediate device).

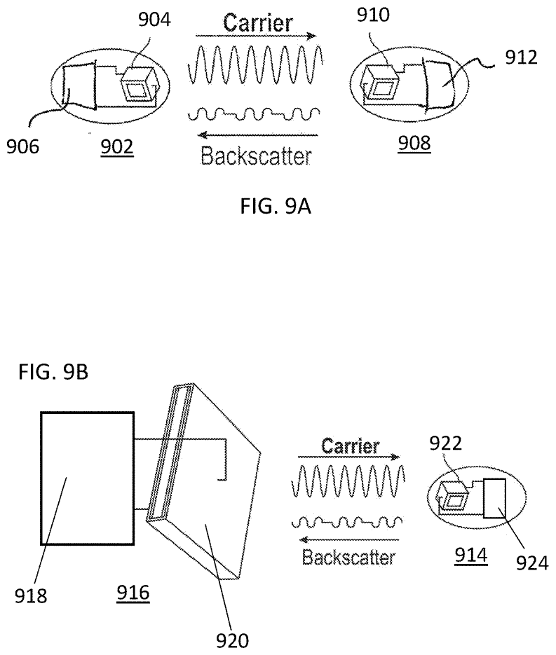

FIG. 9A shows a first implantable device 902 in wireless communication with a second implantable device 908 through ultrasonic waves. The first implantable device 902 generates ultrasonic waves (“carrier waves”) using an ultrasonic transducer 904. Information, such as a detection signal, can be encoded in the ultrasonic carrier waves through an integrated circuit 906. The carrier waves pass through tissue to reach the ultrasonic transducer 910 of the second implantable device 908. The carrier waves cause mechanical vibrations on the ultrasonic transducer 910 (e.g., a bulk piezoelectric transducer, a PUMT, or a CMUT), which generates a voltage across the ultrasonic transducer. The voltage imparts a current flowing through an integrated circuit 912 on the second implantable device 908. The integrated circuit 912 can decode the information encoded in the carrier waves. Additionally, the current flowing through to the ultrasonic transducer 910 causes the transducer on the implantable device to backscatter ultrasonic waves. In some embodiments, the integrated circuit 912 modulates the current flowing through the ultrasonic transducer 912 to encode additional information, and the resulting ultrasonic backscatter waves encode the information. The backscatter waves can be received by the first implantable device 902 or an intermediate device, and can be analyzed to interpret information encoded in the ultrasonic backscatter.

FIG. 9B shows an implantable device 914 in wireless communication with an intermediate device 916 through ultrasonic waves. The intermediate device 916 may be an implantable device or external device, and includes an integrated circuit 918 coupled to an ultrasonic transducer 920. The intermediate device 916 can generate ultrasonic carrier waves through the ultrasonic transducer 920 of the intermediate device, which are received by the ultrasonic transducer 922 of the implantable device. The ultrasonic carrier waves can encode information, such as one or more detection signals or a trigger signal, and the information can be decoded through an integrated circuit 924 of the implantable device 914. In some embodiments, the ultrasonic carrier waves do not encode information, but can be received by the implantable device 914 to supply power to the implantable device 914, or to generate an ultrasonic backscatter waves. The ultrasonic carrier waves received by the ultrasonic transducer 922 generate a current flowing through the ultrasonic transducer 922. The current can be modulated by the integrated circuit 924 of the implantable device 924 to encode information into the ultrasonic backscatter waves. The backscatter waves can be received by the intermediate device 918, or by a different device (i.e., different intermediate device or other implantable device), and can be analyzed to interpret information encoded in the ultrasonic backscatter.

Communication between the devices (i.e., between two or more implantable devices or between an implantable device an intermediate device) can use a pulse-echo method of transmitting and receiving ultrasonic waves. In the pulse-echo method, the device transmits a series of pulses at a predetermined frequency, and then receives backscatter echoes from a separate device. In some embodiments, the pulses are square, rectangular, triangular, sawtooth, or sinusoidal. In some embodiments, the pulses output can be two-level (GND and POS), three-level (GND, NEG, POS), 5-level, or any other multiple-level (for example, if using 24-bit DAC). In some embodiments, the pulses are continuously transmitted by the device during operation. Transducers configured to receive ultrasonic waves and transducers configured to transmit ultrasonic waves can be on the same transducer array or on different transducer arrays of the device. In some embodiments, a transducer on the device can be configured to alternatively transmit or receive the ultrasonic waves. For example, a transducer can cycle between transmitting one or more pulses and a pause period. The transducer is configured to transmit the ultrasonic waves when transmitting the one or more pulses, and can then switch to a receiving mode during the pause period.

In some embodiments, the backscattered ultrasonic waves are digitized by the implantable device or intermediate device. For example, the implantable device can include an oscilloscope or analog-to-digital converter (ADC) and/or a memory, which can digitally encode information in current (or impedance) fluctuations. The digitized current fluctuations, which can encode information, are received by the ultrasonic transducer, which then transmits digitized acoustic waves. The digitized data can compress the analog data, for example by using singular value decomposition (SVD) and least squares-based compression. In some embodiments, the compression is performed by a correlator or pattern detection algorithm. The backscatter signal may go through a series of non-linear transformation, such as 4^{th} order Butterworth bandpass filter rectification integration of backscatter regions to generate a reconstruction data point at a single time instance. Such transformations can be done either in hardware (i.e., hard-coded) or in software.

In some embodiments, the digitized data can include a unique identifier. The unique identifier can be useful, for example, in a system comprising a plurality of implantable devices and/or an implantable device comprising a plurality of electrode pairs. For example, the unique identifier can identify the implantable device of origin when from a plurality of implantable devices, for example when transmitting information from the implantable device (such as a verification signal). In some embodiments, an implantable device comprises a plurality of electrode pairs, which may simultaneously or alternatively emit an electrical pulse by a single implantable device. Different pairs of electrodes, for example, can be configured to emit an electrical pulse in different tissues (e.g., different nerves or different muscles) or in different regions of the same tissue. The digitized circuit can encode a unique identifier to identify and/or verify which electrode pairs emitted the electrical pulse.

In some embodiments, the digitized signal compresses the size of the analog signal. The decreased size of the digitized signal can allow for more efficient reporting of information encoded in the ultrasonic backscatter. By compressing the size of the transmitted information through digitization, potentially overlapping signals can be accurately transmitted.

In some embodiments, an intermediate device communicates with a plurality of implantable devices. This can be performed, for example, using multiple-input, multiple output (MIMO) system theory. For example, communication between the devices using time division multiplexing, spatial multiplexing, or frequency multiplexing. The intermediate device can receive a combined backscatter from the plurality of the implantable devices, which can be deconvoluted, thereby extracting information from each implantable device. In some embodiments, the intermediate device focuses the ultrasonic waves transmitted from a transducer array to a particular implantable device through beam steering. The intermediate device focuses the transmitted ultrasonic waves to a first implantable device, receives backscatter from the first implantable device, focuses transmitted ultrasonic waves to a second implantable device, and receives backscatter from the second implantable device. In some embodiments, the intermediate device transmits ultrasonic waves to a plurality of implantable devices, and then receives ultrasonic waves from the plurality of implantable devices.

In some embodiments, information encoded in the ultrasonic backscatter includes a unique identifier for the implantable device. This can be useful, for example, to ensure the intermediate device is in communication with the correct implantable device when a plurality of implantable devices is implanted in the subject. In some embodiments, the information encoded in the ultrasonic backscatter includes a verification signal that verifies an electrical pulse was emitted by the implantable device. In some embodiments, the information encoded in the ultrasonic backscatter includes an amount of energy stored or a voltage in the energy storage circuit (or one or more capacitors in the energy storage circuit).

### Power Transfer

In some embodiments, the ultrasonic waves received by the implantable device are used to provide power to the implantable device. The ultrasonic waves can be transmitted, for example, by an external device, such as an external intermediate device, which can receive power from another source (e.g., battery, radiofrequency, socket, etc.). The ultrasonic waves can be a different set of ultrasonic waves than those used to wirelessly communicate with the implantable device. The ultrasonic waves can be generated and transmitted, for example, by an external device, such as an external intermediate device, and received by one or more ultrasonic transducers of the one or more implantable devices in the network. Vibrations of the ultrasonic transducer(s) on the implantable device generate a voltage across the electric terminals of the transducer, and current flows through the device, including the integrated circuit. The current can be used to power the integrated circuit of the device, or charge an energy storage circuit within the device (which can include one or more capacitors and/or a battery) if present within the device. The power can be used, for example, to power a computational circuit, one or more detectors on the implantable device, or to emit an electrical pulse, for example to modulate electrophysiological activity of a target nerve.

In some embodiments, the energy storage circuit of the implantable device includes a battery configured to receive the electrical energy from the one or more ultrasonic transducers and power the computational circuit. Inclusion of the battery allows the computational circuit to function without an external power source, including detecting an electrophysiological signal or emitting an electrical pulse to the nerve. The battery can be contained within the body of the implantable device. The battery can be, for example, a rechargeable electrochemical battery. The energy stored by the battery can power the device, for example when the one or more ultrasonic transducers are not receiving ultrasonic waves. The battery can be charged by transmitting ultrasonic waves to the device using an intermediate device which are received by the one or more ultrasonic transducers. The one or more ultrasonic transducers convert the ultrasonic waves into an electrical energy, and are electrically connected to the battery. In this manner, the electrical energy charges the battery of the device.

### Detection Signal

The detection signal detected by the one or more implantable devices is an input (but not necessarily an exclusive input) for determining a trigger signal or updating a dynamic state of a device, which is used by the one or more implantable devices to emit one or more electrical pulses that modulate neural activity of a target nerve. The detection signal can include one or more components, such as a physiological condition or an electrophysiological signal, detected by the one or more implantable devices. The detection signal may be detected by the same implantable device that is configured to emit the electrical pulse, or by a different implantable device.

The detection signal can include one or more detected signals (electrophysiological signal and/or physiological condition) detected simultaneously or at different time points. A time stamp for a detection signal component can be included in the detection signal for analysis to generate the trigger signal.

The detection signal can include one or more physiological conditions detected by the one or more implantable devices, such as one or more of a temperature, a respiratory rate, a strain, a pressure, a pH, a presence of an analyte, or an analyte concentration. Exemplary analytes include glucose and oxygen.

The detection signal can additionally or alternatively include an electrophysiological signal detected from a nerve or subset of nerve fiber (e.g., one or more fascicles within a nerve).

The nerve that transmits the detected electrophysiological signal can be referred to as the “recorded nerve.” In some embodiments, the detected electrophysiological signal component of the detection signal includes, for example, a velocity, a direction, a frequency, an amplitude, a waveform of a compound action potential or a subset of the compound action potential (such as one or more action potential) transmitted by the nerve or a subset of nerve fibers within the nerve. The detected electrophysiological signal component may additionally or alternatively include information related to the subset of nerve fibers from which the electrophysiological signal was detected (that is, a location of the subset of nerve fibers within the nerve). This information can be used by the computational circuit, for example, to select a template detection signal and/or generate the stimulation signal.

The implantable device configured to detect an electrophysiological signal includes a plurality of electrodes in electrical communication with a nerve. Optionally, the electrodes can be located on one or more of the curved members of the implantable device, which wraps at least partially around the nerve and can be configured to detect the electrophysiological signal from a targeted subset of nerve fibers within the nerve. In some embodiments, a curve member wraps substantially around the nerve, such as about 70% or more, about 75% or more, about 80% or more, about 85% or more, about 90% or more, about 95% or more, or about 98% or more around the nerve. The subset of fibers can be, for example, one or more (e.g., 2, 3, 4, or more) fascicles, or a portion of one or more (e.g., 2, 3, 4, or more) fascicles within the nerve. In some embodiments, the subset of nerve fibers comprises or consists of afferent nerve fibers within the nerve, or a subset of afferent nerve fibers within the nerve. In some embodiments, the subset of nerve fibers comprises or consists of efferent nerve fibers within the nerve, or a subset of efferent nerve fibers within the nerve. In some embodiments, the subset of nerve fibers comprises or consists of efferent nerve fibers within two or more fascicles within the nerve or afferent nerve fibers within two or more fascicles within the nerve.

One or more techniques such as computational modeling (e.g., finite element models), inverse source estimation, multipole (e.g., tripole) neural recording, velocity-selective recording, or beamforming can be used to selectively target the subset of nerve fibers. See, for example, Taylor et al., Multiple-electrode nerve cuffs for low-velocity and velocity selective neural recording, Medical & Biological Engineering & Computing, vol. 42, pp. 634-643 (2004); and Wodlinger et al., Localization and Recovery of Peripheral Neural Sources with Beamforming Algorithms, IEEE Transactions on Neural Systems and Rehabilitation Engineering, vol. 17, no. 5, pp. 461-468 (2009). The computational circuit of an implantable device can operate the plurality of electrodes for targeted detection of the electrophysiological signal. Certain nerves may transmit compound electrophysiological signal (or compound action potentials), which is the sum of the electrophysiological signals (or action potentials) simultaneously transmitted by two or more different subsets of nerve fibers. Based on the electrophysiological signal detected by the plurality of electrodes, the computational circuit is able to determine which subset of nerve fibers transmits which electrophysiological signal. In some embodiments, the computational circuit is configured to selectively detect an electrophysiological signal from a targeted subset of nerve fibers using velocity-selective recording, which may be combined with multipolar (e.g., tripolar) recording (which can include any number of tripoles within the plurality of electrodes on one or more curved members). Beamforming can additionally or alternatively be used to detect the electrophysiological signals from the targeted subset of nerve fibers. A portion of or all of the electrode pads of one or more curved members can detect the electrophysiological signal from the nerve, and the computational circuit can determine the cross-sectional location of the transmitted signal within the nerve based on the differences in electrophysiological signal detected by a portion or all of the electrode pads of the one or more curved members.

Information related to the detection signal or detection signal components detected by an implantable device can be stored on a non-transitory memory of the device and/or wirelessly transmitted from the implantable device. The information transmitted from the implantable device can be received by one or more intermediate devices and/or one or more other implantable devices of the network.

### Detection Signal Analysis, Trigger Signal Generation, and Dynamic States of the Implantable Devices

The information related to the detection signal can be analyzed to generate a trigger signal for an implantable device, which is used as a basis for generating a stimulation signal and emit one or more electrical pulse that modulates activity of a target nerve. The trigger signal may be the output of a feedforward process, or may be a dynamic state of an implantable device in a recurrent neural network process. For example, in some embodiments, a trigger signal is generated by analyzing information inputs (such one or more detection signal detected by one or more implantable devices, or a timestamp and/or location associated with the detection signal). In some embodiments, an implantable device has a dynamic state, which may be updated based on information inputs (such one or more detection signal detected by one or more implantable devices, a timestamp and/or location associated with the detection signal, and/or a dynamic state of another implantable device) and a previous dynamic state of the implantable device.

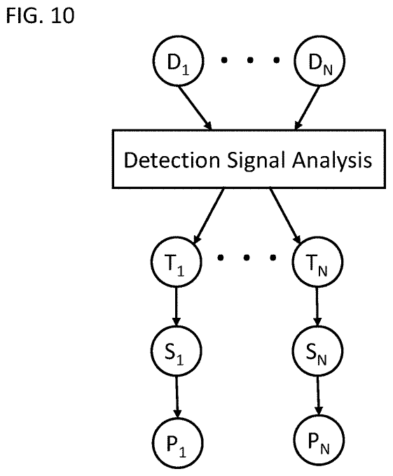

An exemplary process is illustrated in FIG. 10. D₁ through D_{N} represent one or more components of the detection signal information based on the signal detected by the one or more implantable devices are used as inputs for analysis. The one or more detection signal components can be from the same device or different devices. The information related to the detection signal is analyzed to generate one or more trigger signals, T₁ through T_{N}. The information related to the detection signal can be analyzed by an implantable device that detects one or more of the detection signals, an intermediate device, or an implantable device that emits the electrical pulse. For example, an implantable device can detect one or more detection signal components, analyze the detection signal components to generate one or more trigger signals, and wirelessly transmit the trigger signals to one or more other implantable devices. In another example, information related to the detection signal can be wirelessly transmitted to an intermediate device, which analyzes the information to generate one or more trigger signals that are wirelessly transmitted to one or more implantable devices. If the network includes a plurality of implantable devices, the intermediate device can receive the information related to the detection signal (or detection signal components) from the plurality of implantable devices, and analyze the information. Wirelessly transmitted information based on the detection signal components can include a unique identifier so that the source location of the information can be recognized during the analysis. A wirelessly transmitted trigger signal can also include a unique identifier such that an implantable device can correctly associate the trigger signal with the implantable device. In some embodiments, the information related to the detection signal is received by an implantable device, which analyzes the detection signal to generate a trigger signal for that device.

Once an implantable device obtains a trigger signal (either by wirelessly receiving the trigger signals from a separate device, such as an intermediate device or an another implantable device, or by generating the trigger signal by analyzing the detection signal), the implantable device can generate a stimulation signal (e.g., S₁ for a first implantable device, and S_{N} for an Nth implantable device). However, the trigger signal could be a null signal, indicating that no stimulation signal should be generated. The stimulation signal causes the stimulation circuit of the implantable device to generate an electrical pulse (P₁ for a first implantable device, and P_{N} for an Nth implantable device) that modulates neural activity of a target nerve.

The analysis of the information related to the detection signal to generate the trigger signal can include, for example, identifying a modulation of the detection signal (such as a modulation of the detected electrophysiological signal, the detected physiological condition, or both), which can act as a trigger for generation of the stimulation signal. The modulation of the electrophysiological signal can indicate, for example, a compound action potential or a component of the compound action potential (e.g., one or more action potentials) that is being transmitted by the nerve. The trigger signal can be generated using a mathematical relationship between the detection signal and the stimulation signal. The mathematical relationship can be, for example, determined by using machine learning or can be a pre-selected mathematical relationship. In some embodiments, the computational circuit uses a digital logic, an analog logic, an artificial neural network, a convolutional neural network (CNN) or neuromorphic computing.

In some embodiments, generating the trigger signal can include comparing the detection signal to a template detection signal, and the stimulation signal is generated based on the variance or similarity between the detection signal and the template detection signal. One or more template detection signals can be stored, for example, on a non-transitory memory in the body of the device. The computational circuit can use, for example, a digital logic, an analog logic, an artificial neural network, a convolutional neural network (CNN), or neuromorphic computing to detect the variance or similarity between the detected electrophysiological signal and the template electrophysiological signal.

### Neuromodulation

The trigger signal transmitted obtained by an implantable device configured to emit an electrical pulse can include instructions for generating the stimulation and emitting the electrical pulse, and may include instructions for one or more pulse characteristics, such as the type of pulse (e.g., direct current pulse or alternating current pulse), a number pulses, a dwell time between pulses, a pulse frequency, a pulse amplitude, a pulse shape, or a pulse voltage. As discussed above, the trigger signal is based on information related to the detection signal, and can include one or more components detected by one or more implantable devices from a different device, but may also include one or more components detected by the same implantable device.

The computational circuit generates a stimulation signal to operate the stimulation circuit, and can include information about the electrical pulse to be emitted by the device. The stimulation circuit may be directly derived from the trigger signal, or may be obtained from a lookup table based on the trigger signal. For example, in some embodiments, one or more template pulses are stored on a non-transitory memory within the device, and the computational circuit can generate the stimulation signal by retrieving a template pulse from the non-transitory memory using the detection signal.

The implantable device that emits the electrical pulse includes two or more electrodes configured to emit an electrical pulse or an electrical pulse train (i.e., a plurality of electrical pulses, which may be the same or different) that modulates the activity of a target nerve. The electrodes can be positioned at different locations along the length of the nerve or around the circumference of the nerve, and are configured to emit an electrical pulse at the different positions. The electrical pulse emitted by the two or more different electrodes may be the same or different, and may be targeted to the same or different subset of curved members within the nerve. For example, a first plurality of electrodes can emit an electrical pulse train configured to block transmission of an electrophysiological signal by a first subset of nerve subset of nerve fibers, and a second plurality of electrodes can be configured to emit an electrical pulse or train that stimulates a second subset of nerve fibers. In some embodiments, the first subset of nerve fibers can be, for example, efferent nerve fibers, while the second subset of nerve fibers is afferent nerve fibers. In other embodiments the first subset of nerve fibers are afferent nerve fibers, and the second subset of nerve fibers are efferent nerve fibers. By blocking transmission of an electrophysiological signal in a first subset of nerve fibers and stimulating a second subset of nerve fibers, off-target effects of the stimulation are minimized. In another example, the one or more electrodes within the first plurality of electrode pads on the first curved member and one or more electrodes within the second plurality of electrode pads on the second curved members can be operated for bipolar stimulation along the length of the nerve. In a further example, the plurality of electrodes on the first curved member and the plurality of electrodes on the second curved member can each emit a coordinated electrical pulse (that is, the electrical pulses emitted by the separate pluralities of electrodes are coordinated with each other), which can be used for specific focal stimulation.

The one or more electrical pulses emitted by the implantable device may include a targeted electrical pulse to a subset of nerve fibers within the nerve by selectively activating one or more electrode pads within the plurality of electrode pads on the curved member. The computational circuit of the device can operate the stimulation circuit to selectively activate the electrodes (i.e., via the stimulation signal). Selective activation can include, for example, activating a portion of the electrodes and/or differentially activating all or a portion of the electrodes. The plurality of electrodes can therefore be operated to steer the electrical pulse emitted by the plurality of electrode pads to the target subset of nerve fibers. Techniques such as electrical field interference and/or multipolar stimulation (e.g., tripolar stimulation) can be used to target the electrical pulse to the subset of nerve fibers within the nerve. See, for example, Grossman, et al., Noninvasive Deep Brain Stimulation via Temporally Interfering Electrical Fields, Cell, vol. 169, pp. 1029-1041 (2017). The electrode pads with one or more curved members can be selectively activated by the computational circuit to target the emitted electrical pulse to the subset of nerve fibers. The subset of nerve fibers targeted by the electrical pulse emitted by the device can be, for example, one or more (e.g., 2, 3, 4, or more) fascicles, or a portion of one or more (e.g., 2, 3, 4, or more) fascicles within the nerve. In some embodiments, the subset of nerve fibers comprises or consists of afferent nerve fibers within the nerve, or a subset of afferent nerve fibers within the nerve. In some embodiments, the subset of nerve fibers comprises or consists of efferent nerve fibers within the nerve, or a subset of efferent nerve fibers within the nerve. In some embodiments, the subset of nerve fibers comprises or consists of efferent nerve fibers within two or more fascicles within the nerve or afferent nerve fibers within two or more fascicles within the nerve.

### EXEMPLARY EMBODIMENTS

The following embodiments are exemplary and should not be considered to limit the invention.

Embodiment 1. A method of modulating neural activity using an implantable device network, comprising:

(a) detecting, at one or more implantable devices in a first set of one or more implantable devices, a detection signal comprising one or more electrophysiological signals transmitted by a recorded nerve or one or more physiological conditions;

(b) wirelessly transmitting, from the one or more implantable devices in the first set of one or more implantable devices, information related to the detection signal;

(c) wirelessly receiving, at one or more implantable devices in a second set of one or more implantable devices, the information related to the detection signal; and

(d) determining whether to emit, from one or more implantable devices in the second set of one or more implantable devices, one or more electrical pulses configured to modulate neural activity of one or more target nerves based on at least the received information related to the detection signal.

Embodiment 2. The method of embodiment 1, comprising emitting, at the one or more implantable devices in the second set of one or more implantable devices, the one or more electrical pulses.

Embodiment 3. The method of embodiment 2, comprising determining one or more pulse characteristics of the one or more electrical pulses emitted from the one or more implantable devices in the second set of one or more implantable devices in the second set of one or more implantable devices.

Embodiment 4. The method of any one of embodiments 1-3, comprising:

wirelessly transmitting, from the one or more implantable devices in the second set of one or more implantable devices, information related to the one or more implantable devices in the second set of one or more implantable devices;

wirelessly receiving, at the one or more implantable devices in the first set of one or more implantable devices, the information related to the one or more implantable devices in the second set of one or more implantable devices; and

determining whether to emit, from one or more implantable devices in the first set of one or more implantable devices, one or more electrical pulses configured to modulate neural activity of one or more additional target nerves based on at least the information related to the one or more implantable devices in the second set of one or more implantable devices.

Embodiment 5. The method of embodiment 4, comprising emitting, at the one or more implantable devices in the first set of one or more implantable devices, the one or more electrical pulses configured to modulate neural activity of the one or more additional target nerves.

Embodiment 6. The method of embodiment 4 or 5, wherein determining whether to emit the one or more electrical pulses configured to modulate neural activity of the one or more additional nerves comprises updating a dynamic state of the one or more implantable devices in the first set of one or more implantable devices.

Embodiment 7. The method of any one of embodiments 4-6, wherein the information related to the one or more implantable devices in the second set of one or more implantable devices wirelessly transmitted by the one or more implantable devices in the second set of one or more implantable devices comprises information related to a detection signal detected by the one or more implantable devices in the second set of one or more implantable devices.

Embodiment 8. The method of any one of embodiments 4-7, wherein the information related to the one or more implantable devices in the second set of one or more implantable devices wirelessly transmitted by the one or more implantable devices in the second set of one or more implantable devices comprises information related to a dynamic state of one or more of the implantable devices in the second set of one or more implantable devices.

Embodiment 9. The method of any one of embodiments 4-8, wherein the information related to the one or more implantable devices in the second set of one or more implantable devices wirelessly transmitted by the one or more implantable devices in the second set of one or more implantable devices comprises information related to the one or more electrical pulses emitted by the one or more implantable devices in the second set of one or more implantable devices.

Embodiment 10. The method of any one of embodiments 1-9, wherein determining whether to emit the one or more electrical pulses from the one or more implantable devices in the second set of one or more implantable devices comprises implementing a feedforward neural network process.

Embodiment 11. The method of any one of embodiments 1-9, wherein determining whether to emit the one or more electrical pulses from the one or more implantable devices in the second set of one or more implantable devices comprises updating a dynamic state of one or more implantable devices in the second set of one or more implantable devices.

Embodiment 12. The method of any one of embodiments 1-11, wherein a determination of whether to emit the one or more electrical pulses from the one or more implantable devices in the second set of one or more implantable devices is further based a detection signal detected by the one or more implantable devices in the second set of one or more implantable devices.

Embodiment 13. The method of any one of embodiments 1-12, wherein a determination of whether to emit the one or more electrical pulses from the one or more implantable devices in the second set of one or more implantable devices is made by an implantable device in the first set of one or implantable devices.

Embodiment 14. The method of any one of embodiments 1-12, wherein a determination of whether to emit the one or more electrical pulses from the one or more implantable devices in the second set of one or more implantable devices is made by an implantable device in the second set of one or implantable devices.

Embodiment 15. The method of any one of embodiments 1-14, comprising directly transmitting the information related to the detection signal detected by the one or more implantable devices in the first set of one or more implantable devices from one or more of the implantable devices in the first set of one or more implantable devices to one or more of the implantable devices in the second set of one or more implantable devices.

Embodiment 16. The method of any one of embodiments 1-14, comprising transmitting the information related to the detection signal detected by the one or more implantable devices in the first set of one or more implantable devices from one or more of the implantable devices in the first set of one or more implantable devices to one or more of the implantable devices in the second set of one or more implantable devices through one or more intermediate devices.

Embodiment 17. The method of embodiment 16, wherein a determination of whether to emit the one or more electrical pulses from the one or more implantable devices in the second set of one or more implantable devices is made by the one or more intermediate devices.

Embodiment 18. The method of any one of embodiments 1-17, wherein the first set of one or more implantable devices comprises two or more implantable devices.

Embodiment 19. The method of any one of embodiments 1-18, wherein the second set of one or more implantable devices comprises two or more implantable devices.

Embodiment 20. The method of any one of embodiments 1-19, comprising generating a stimulation signal based on at least the received information related to the detection signal, wherein the stimulation signal drives the one or more electrical pulses emitted by the one or more implantable devices.

Embodiment 21. The method of any one of embodiments 1-20, wherein the detection signal comprises the one or more physiological conditions.

Embodiment 22. The method of embodiment 21, wherein the one or more physiological conditions comprises a temperature, a respiratory rate, a strain, a pressure, a pH, a presence of an analyte, or an analyte concentration.

Embodiment 23. The method of any one of embodiments 1-22, wherein the detection signal comprises the one or more electrophysiological signals.

Embodiment 24. The method of any one of embodiments 1-23, wherein the information related to the detection signal comprises:

a timestamp of the electrophysiological signal or the physiological condition; or

a direction, a velocity, a frequency, an amplitude, or a waveform of a compound action potential or a portion thereof within the electrophysiological signal.

Embodiment 25. The method of any one of embodiments 1-24, wherein:

one of the one or more implantable devices in the first set of one or more implantable devices detects the electrophysiological signal from a first nerve locus; and

one of the one or more implantable devices in the second set of one or more implantable devices emits the electrical pulse configured to modulate neural activity of a second nerve locus, wherein the first nerve locus and the second nerve locus are different positions on the same nerve or different nerves.

Embodiment 26. The method of embodiment 25, wherein the first nerve locus and the second locus are different nerves connected through a nerve network.

Embodiment 27. The method of embodiment 25, wherein the first nerve locus and the second nerve locus are the same nerve.

Embodiment 28. The method of any one of embodiment 25-27, wherein the electrophysiological signal detected by the one of the one or more implantable devices in the first set of one or more implantable devices is transmitted by a subset of nerve fibers within the first nerve locus.

Embodiment 29. The method of embodiment 28, wherein the subset of nerve fibers comprises one or more fascicles within the first nerve locus.

Embodiment 30. The method of embodiment 28 or 29, wherein the subset of nerve fibers comprises one or more afferent nerve fibers.

Embodiment 31. The method of embodiment 28 or 29, wherein the subset of nerve fibers comprises one or more efferent nerve fibers.

Embodiment 32. The method of any one of embodiments 28-31, wherein the subset of nerve fibers comprises two or more nerve fibers in different fascicles within the nerve.

Embodiment 33. The method of any one of embodiments 1-32, wherein wirelessly transmitting the information related to the detection signal from the one or more implantable devices in the first set of one or more implantable devices comprises actively transmitting from the one or more implantable devices in the first set of one or more implantable devices ultrasonic waves that encode the information related to the detection signal.

Embodiment 34. The method of any one of embodiments 1-32, wherein wirelessly transmitting the information related to the detection signal from the one or more implantable devices in the first set of one or more implantable devices comprises:

receiving ultrasonic waves at the one or more implantable devices in the first set of one or more implantable devices; and

backscattering the ultrasonic waves from the one or more implantable devices in the first set of one or more implantable devices, wherein the backscattered ultrasonic waves encode the information related to the detection signal.

Embodiment 35. The method of any one of embodiments 1-33, wherein the information related to the detection signal received at the one or more implantable devices in the second set of one or more implantable devices is encoded in ultrasonic waves received by the one or more implantable devices in the second set of one or more implantable devices.

Embodiment 36. The method of any one of embodiments 33-35, wherein wirelessly transmitting, from the one or more implantable devices in the first set of one or more implantable devices, the information related to the detection signal detected by the one or more implantable devices in the first set of one or more implantable devices comprises:

receiving, at an intermediate device, the ultrasonic waves encoding the information related to the detection signal actively transmitted or backscattered by the one or more implantable devices in the first set of one or more implantable device;

actively transmitting, from the intermediate device, additional ultrasonic waves that encode the information related to the detection signal; and

receiving, at the one or more implantable devices in the second set of one or more implantable devices, the additional ultrasonic waves actively transmitted from the intermediate device.

Embodiment 37. The method of embodiment 36, wherein the intermediate device is an external device.

Embodiment 38. The method of any one of embodiments 1-37, wherein the one or more implantable devices in the first set of one or more implantable devices or the one or more implantable devices in the second set of one or more implantable devices are powered using powering ultrasonic waves.

Embodiment 39. The method of embodiment 38, wherein the powering ultrasonic waves are transmitted by an intermediate device.

Embodiment 40. The method of any one of embodiments 1-39, wherein the electrical pulse emitted by the one or more implantable devices in the second set of one or more implantable devices is emitted to a targeted subset of nerve fibers within the target nerve.

Embodiment 41. The method of embodiment 40, wherein the targeted subset of nerve fibers comprises one or more fascicles within the first nerve.

Embodiment 42. The method of embodiment 40 or 41, wherein the targeted subset of nerve fibers comprises one or more afferent nerve fibers.

Embodiment 43. The method of embodiment 40 or 41, wherein the targeted subset of nerve fibers comprises one or more efferent nerve fibers.

Embodiment 44. The method of any one of embodiments 40-43, wherein the targeted subset of nerve fibers comprises two or more nerve fibers in different fascicles within the target nerve.

Embodiment 45. The method of any one of embodiments 1-44, wherein the one or more implantable devices in the second set of one or more implantable devices emits the electrical pulse to a fibrous tissue comprising the target nerve.

Embodiment 46. The method of any one of embodiments 1-45, wherein the target nerve is a vagus nerve, a spinal cord, a splenic nerve, a mesenteric nerve, a sciatic nerve, a tibial nerve, a celiac ganglion, a sacral nerve, a renal nerve, an occipital nerve, or an adrenal nerve.

Embodiment 47. The method of any one of embodiments 1-46, wherein the target nerve is a peripheral nerve.

Embodiment 48. The method of any one of embodiments 1-47, wherein the recorded nerve is a vagus nerve, a spinal cord, a splenic nerve, a mesenteric nerve, a sciatic nerve, a tibial nerve, a celiac ganglion, a sacral nerve, a renal nerve, an occipital nerve, or an adrenal nerve.

Embodiment 49. The method of any one of embodiments 1-48, wherein the recorded nerve is a peripheral nerve.

Embodiment 50. A device network for modulating neural activity of a target nerve, comprising:

(a) one or more implantable devices in a first set of one or more implantable devices, comprising:
a sensor for detecting a detection signal, comprising an electrophysiological signal transmitted by a nerve or a physiological condition,
an ultrasonic transducer configured to actively transmit or backscatter ultrasonic waves, wherein the ultrasonic waves encode information related to the detection signal, and
a control circuit electrically coupled to the sensor and the ultrasonic transducer; and

(b) one or more implantable devices in a second set of one or more implantable devices, comprising:
a plurality of electrodes configured to emit an electrical pulse to a target nerve,
an ultrasonic transducer configured to receive ultrasonic waves encoding information related to the detection signal, and
a control circuit configured to extract the information related to the detection signal from the ultrasonic waves, and to operate the plurality of electrodes to emit the electrical pulse based on the information related to the detection signal;

wherein the one or more implantable devices in the first set of one or more implantable devices and the one or more implantable devices in the second set of one or more implantable devices are configured to wirelessly transmit information from the one or more implantable devices in the first set of one or more implantable devices to the one or more implantable devices in the second set of one or more implantable devices.

Embodiment 51. The device network of embodiment 50, wherein the one or more implantable devices in the first set of one or more implantable devices and the one or more implantable devices in the second set of one or more implantable devices are configured to wirelessly transmit information from the one or more implantable devices in the second set of one or more implantable devices to the one or more implantable devices in the first set of one or more implantable devices.

Embodiment 52. The device network of embodiment 50 or 51, wherein the device network comprises two or more implantable devices in the first set of one or more implantable devices.

Embodiment 53. The device network of any one of embodiments 50-52, wherein the device network comprises two or more implantable devices in the second set of one or more implantable devices.

Embodiment 54. The device network of any one of embodiments 50-53, wherein the control circuit of the one or more implantable devices in the first set of one or more implantable devices or the second set of one or more implantable devices is configured to determine whether to emit one or more electrical pulses from the one or more implantable devices based at least on information wirelessly received by the one or more implantable devices.

Embodiment 55. The device network of embodiment 54, wherein the control circuit of the one or more implantable devices in the first set of one or more implantable devices or the second set of one or more implantable devices is configured to select one or more pulse characteristics of the one or more electrical pulses.

Embodiment 56. The device network of embodiment 54 or 55, wherein determining whether to emit an electrical pulse comprises updating a dynamic state of the one or more implantable devices.

Embodiment 57. The device network of any one of embodiments 50-56, wherein the one or more implantable devices in the second set of one or more implantable devices further comprise a sensor for detecting an electrophysiological signal transmitted by a nerve or a physiological condition.

Embodiment 58. The device network of any one of embodiments 50-57, further comprising one or more intermediate devices comprising an ultrasonic transducer, wherein the one or more intermediate devices are configured to:

wirelessly receive the information from the one or more implantable devices in the first set of one or more implantable devices through ultrasonic waves, and wirelessly transmit the information to the one or more implantable devices in the second set of one or more implantable devices through ultrasonic waves.

Embodiment 59. The device network of embodiment 58, wherein the one or more intermediate devices are further configured to:

wirelessly receive the information from the one or more implantable devices in the second set one or more implantable devices of implantable devices through ultrasonic waves, and

wirelessly transmit the information to the one or more implantable devices in the first set of one or more implantable devices through ultrasonic waves.

Embodiment 60. The device network of embodiment 58 or 59, wherein the one or more intermediate devices are configured to:

actively transmit ultrasonic waves to the one or more implantable device in the first set of one or more implantable devices;

receive backscattered ultrasonic waves that encode the information related to the detection signal detected by the sensor; and

actively transmit ultrasonic waves that encode the information related to the detection signal to the one or more implantable devices in the second set of one or more implantable devices.

Embodiment 61. The device network of any one of embodiments 58-60, wherein the intermediate device comprises a control circuit configured to:

extract the information related to the detection signal from the ultrasonic waves received by the intermediate device, and

determine whether one or more electrical pulses should be emitted from the one or more implantable devices in the second set of one or more implantable devices based at least on information wirelessly received by the one or more implantable devices in the first set of one or more implantable devices;

wherein the information related to the detection signal encoded in the ultrasonic waves transmitted from the intermediate device to the one or more implantable devices in the second set of one or more implantable devices comprises instructions to emit the one or more electrical pulses, and

wherein the control circuit of the one or more implantable devices in the second set of one or more implantable devices is configured to operate the plurality of electrodes to emit the electrical pulse based on the instructions.

Embodiment 62. The device network of embodiment 61, wherein the instructions to emit the one or more electrical pulses comprises instructions for one or more pulse characteristics of the one or more electrical pulses.

Embodiment 63. The device network of any one of embodiments 50-60, wherein:

the control circuit of the one or more implantable devices in the first set of one or more implantable devices is configured to determine whether one or more electrical pulses should be emitted from one or more implantable devices in the second set of one or more implantable devices based on at least the detection signal detected by the sensor of the one or more implantable devices in the first set of one or more implantable devices;

wherein the information related to the detection signal encoded in the ultrasonic waves actively transmitted or backscattered by the ultrasonic transducer of the one or more implantable devices in the first set of one or more implantable devices comprises instructions to emit the one or more electrical pulses; and

wherein the control circuit of the one or more implantable devices in the second set of one or more implantable devices is configured to operate the plurality of electrodes to emit the electrical pulse based on the instructions.

Embodiment 64. The device network of embodiment 63, wherein the instructions to emit the one or more electrical pulses comprises instructions for one or more pulse characteristics of the one or more electrical pulses.

Embodiment 65. The device network of any one of embodiments 50-60, wherein the control circuit of the one or more implantable devices in the second set of one or more implantable devices is configured to determine whether one or more electrical pulses should be emitted from the one or more implantable devices in the second set of one or more implantable devices based on at least the information related to the detection signal, and operate the plurality of electrodes to emit the electrical pulse based on the determination.

Embodiment 66. The device network of embodiment 65, wherein the control circuit is further configured to select one or more pulse characteristics of the one or more electrical pulses.

Embodiment 67. The device network of any one of embodiments 50-66, wherein the sensor comprises a plurality of electrodes configured to detect the electrophysiological signal.

Embodiment 68. The device network of any one of embodiments 48-67, wherein the sensor is configured to detect the physiological condition.

Embodiment 69. The device network of embodiment 68, wherein the physiological condition is a temperature, a respiratory rate, a strain, a pressure, a pH, a presence of an analyte, or an analyte concentration.

Embodiment 70. The device network of any one of embodiments 50-69, wherein the one or more implantable devices in the first set of one or more implantable devices comprises a first sensor configured to detect the physiological condition, and a second sensor comprising a plurality of electrodes configured to detect the electrophysiological signal.

Embodiment 71. The device network of any one of embodiments 50-70, wherein the ultrasonic transducer of the one or more implantable devices in the first set of one or more implantable devices or the ultrasonic transducer of the one or more implantable devices in the second set of one or more implantable devices is configured to receive ultrasonic waves that power the one or more implantable devices.

Although examples of this disclosure have been fully described with reference to the accompanying drawings, it is to be noted that various changes and modifications will become apparent to those skilled in the art. Such changes and modifications are to be understood as being included within the scope of examples of this disclosure as defined by the appended claims.

What is claimed is:

1. A method of modulating neural activity using an implantable device network, comprising:
(a) detecting, at one or more implantable devices in a first set of one or more implantable devices, a detection signal comprising one or more electrophysiological signals transmitted by a recorded nerve or one or more physiological conditions;
(b) wirelessly transmitting, from the one or more implantable devices in the first set of one or more implantable devices, information related to the detection signal;
(c) wirelessly receiving, at one or more implantable devices in a second set of one or more implantable devices, the information related to the detection signal; and
(d) determining whether to emit, from one or more implantable devices in the second set of one or more implantable devices, one or more electrical pulses configured to modulate neural activity of one or more target nerves based on at least the received information related to the detection signal.

2. The method of claim 1, comprising emitting, at the one or more implantable devices in the second set of one or more implantable devices, the one or more electrical pulses.

3. The method of claim 2, comprising determining one or more pulse characteristics of the one or more electrical pulses emitted from the one or more implantable devices in the second set of one or more implantable devices in the second set of one or more implantable devices.

4. The method of any one of claims 1-3, comprising:
wirelessly transmitting, from the one or more implantable devices in the second set of one or more implantable devices, information related to the one or more implantable devices in the second set of one or more implantable devices;
wirelessly receiving, at the one or more implantable devices in the first set of one or more implantable devices, the information related to the one or more implantable devices in the second set of one or more implantable devices; and
determining whether to emit, from one or more implantable devices in the first set of one or more implantable devices, one or more electrical pulses configured to modulate neural activity of one or more additional target nerves based on at least the information related to the one or more implantable devices in the second set of one or more implantable devices.

5. The method of claim 4, comprising emitting, at the one or more implantable devices in the first set of one or more implantable devices, the one or more electrical pulses configured to modulate neural activity of the one or more additional target nerves.

6. The method of claim 4 or 5, wherein determining whether to emit the one or more electrical pulses configured to modulate neural activity of the one or more additional nerves comprises updating a dynamic state of the one or more implantable devices in the first set of one or more implantable devices.

7. The method of any one of claims 4-6, wherein the information related to the one or more implantable devices in the second set of one or more implantable devices wirelessly transmitted by the one or more implantable devices in the second set of one or more implantable devices comprises information related to a detection signal detected by the one or more implantable devices in the second set of one or more implantable devices.

8. The method of any one of claims 4-7, wherein the information related to the one or more implantable devices in the second set of one or more implantable devices wirelessly transmitted by the one or more implantable devices in the second set of one or more implantable devices comprises information related to a dynamic state of one or more of the implantable devices in the second set of one or more implantable devices.

9. The method of any one of claims 4-8, wherein the information related to the one or more implantable devices in the second set of one or more implantable devices wirelessly transmitted by the one or more implantable devices in the second set of one or more implantable devices comprises information related to the one or more electrical pulses emitted by the one or more implantable devices in the second set of one or more implantable devices.

10. The method of any one of claims 1-9, wherein determining whether to emit the one or more electrical pulses from the one or more implantable devices in the second set of one or more implantable devices comprises implementing a feedforward neural network process.

11. The method of any one of claims 1-9, wherein determining whether to emit the one or more electrical pulses from the one or more implantable devices in the second set of one or more implantable devices comprises updating a dynamic state of one or more implantable devices in the second set of one or more implantable devices.

12. The method of any one of claims 1-11, wherein a determination of whether to emit the one or more electrical pulses from the one or more implantable devices in the second set of one or more implantable devices is further based a detection signal detected by the one or more implantable devices in the second set of one or more implantable devices.

13. The method of any one of claims 1-12, wherein a determination of whether to emit the one or more electrical pulses from the one or more implantable devices in the second set of one or more implantable devices is made by an implantable device in the first set of one or implantable devices.

14. The method of any one of claims 1-12, wherein a determination of whether to emit the one or more electrical pulses from the one or more implantable devices in the second set of one or more implantable devices is made by an implantable device in the second set of one or implantable devices.

15. The method of any one of claims 1-14, comprising directly transmitting the information related to the detection signal detected by the one or more implantable devices in the first set of one or more implantable devices from one or more of the implantable devices in the first set of one or more implantable devices to one or more of the implantable devices in the second set of one or more implantable devices.

16. The method of any one of claims 1-14, comprising transmitting the information related to the detection signal detected by the one or more implantable devices in the first set of one or more implantable devices from one or more of the implantable devices in the first set of one or more implantable devices to one or more of the implantable devices in the second set of one or more implantable devices through one or more intermediate devices.

17. The method of claim 16, wherein a determination of whether to emit the one or more electrical pulses from the one or more implantable devices in the second set of one or more implantable devices is made by the one or more intermediate devices.

18. The method of any one of claims 1-17, wherein the first set of one or more implantable devices comprises two or more implantable devices.

19. The method of any one of claims 1-18, wherein the second set of one or more implantable devices comprises two or more implantable devices.

20. The method of any one of claims 1-19, comprising generating a stimulation signal based on at least the received information related to the detection signal, wherein the stimulation signal drives the one or more electrical pulses emitted by the one or more implantable devices.

21. The method of any one of claims 1-20, wherein the detection signal comprises the one or more physiological conditions.

22. The method of claim 21, wherein the one or more physiological conditions comprises a temperature, a respiratory rate, a strain, a pressure, a pH, a presence of an analyte, or an analyte concentration.

23. The method of any one of claims 1-22, wherein the detection signal comprises the one or more electrophysiological signals.

24. The method of any one of claims 1-23, wherein the information related to the detection signal comprises:
a timestamp of the electrophysiological signal or the physiological condition; or
a direction, a velocity, a frequency, an amplitude, or a waveform of a compound action potential or a portion thereof within the electrophysiological signal.

25. The method of any one of claims 1-24, wherein:
one of the one or more implantable devices in the first set of one or more implantable devices detects the electrophysiological signal from a first nerve locus; and
one of the one or more implantable devices in the second set of one or more implantable devices emits the electrical pulse configured to modulate neural activity of a second nerve locus, wherein the first nerve locus and the second nerve locus are different positions on the same nerve or different nerves.

26. The method of claim 25, wherein the first nerve locus and the second locus are different nerves connected through a nerve network.

27. The method of claim 25, wherein the first nerve locus and the second nerve locus are the same nerve.

28. The method of any one of claim 25-27, wherein the electrophysiological signal detected by the one of the one or more implantable devices in the first set of one or more implantable devices is transmitted by a subset of nerve fibers within the first nerve locus.

29. The method of claim 28, wherein the subset of nerve fibers comprises one or more fascicles within the first nerve locus.

30. The method of claim 28 or 29, wherein the subset of nerve fibers comprises one or more afferent nerve fibers.

31. The method of claim 28 or 29, wherein the subset of nerve fibers comprises one or more efferent nerve fibers.

32. The method of any one of claims 28-31, wherein the subset of nerve fibers comprises two or more nerve fibers in different fascicles within the nerve.

33. The method of any one of claims 1-32, wherein wirelessly transmitting the information related to the detection signal from the one or more implantable devices in the first set of one or more implantable devices comprises actively transmitting from the one or more implantable devices in the first set of one or more implantable devices ultrasonic waves that encode the information related to the detection signal.

34. The method of any one of claims 1-32, wherein wirelessly transmitting the information related to the detection signal from the one or more implantable devices in the first set of one or more implantable devices comprises:
receiving ultrasonic waves at the one or more implantable devices in the first set of one or more implantable devices; and
backscattering the ultrasonic waves from the one or more implantable devices in the first set of one or more implantable devices, wherein the backscattered ultrasonic waves encode the information related to the detection signal.

35. The method of any one of claims 1-33, wherein the information related to the detection signal received at the one or more implantable devices in the second set of one or more implantable devices is encoded in ultrasonic waves received by the one or more implantable devices in the second set of one or more implantable devices.

36. The method of any one of claims 33-35, wherein wirelessly transmitting, from the one or more implantable devices in the first set of one or more implantable devices, the information related to the detection signal detected by the one or more implantable devices in the first set of one or more implantable devices comprises:
receiving, at an intermediate device, the ultrasonic waves encoding the information related to the detection signal actively transmitted or backscattered by the one or more implantable devices in the first set of one or more implantable device;
actively transmitting, from the intermediate device, additional ultrasonic waves that encode the information related to the detection signal; and
receiving, at the one or more implantable devices in the second set of one or more implantable devices, the additional ultrasonic waves actively transmitted from the intermediate device.

37. The method of claim 36, wherein the intermediate device is an external device.

38. The method of any one of claims 1-37, wherein the one or more implantable devices in the first set of one or more implantable devices or the one or more implantable devices in the second set of one or more implantable devices are powered using powering ultrasonic waves.

39. The method of claim 38, wherein the powering ultrasonic waves are transmitted by an intermediate device.

40. The method of any one of claims 1-39, wherein the electrical pulse emitted by the one or more implantable devices in the second set of one or more implantable devices is emitted to a targeted subset of nerve fibers within the target nerve.

41. The method of claim 40, wherein the targeted subset of nerve fibers comprises one or more fascicles within the first nerve.

42. The method of claim 40 or 41, wherein the targeted subset of nerve fibers comprises one or more afferent nerve fibers.

43. The method of claim 40 or 41, wherein the targeted subset of nerve fibers comprises one or more efferent nerve fibers.

44. The method of any one of claims 40-43, wherein the targeted subset of nerve fibers comprises two or more nerve fibers in different fascicles within the target nerve.

45. The method of any one of claims 1-44, wherein the one or more implantable devices in the second set of one or more implantable devices emits the electrical pulse to a fibrous tissue comprising the target nerve.

46. The method of any one of claims 1-45, wherein the target nerve is a vagus nerve, a spinal cord, a splenic nerve, a mesenteric nerve, a sciatic nerve, a tibial nerve, a celiac ganglion, a sacral nerve, a renal nerve, an occipital nerve, or an adrenal nerve.

47. The method of any one of claims 1-46, wherein the target nerve is a peripheral nerve.

48. The method of any one of claims 1-47, wherein the recorded nerve is a vagus nerve, a spinal cord, a splenic nerve, a mesenteric nerve, a sciatic nerve, a tibial nerve, a celiac ganglion, a sacral nerve, a renal nerve, an occipital nerve, or an adrenal nerve.

49. The method of any one of claims 1-48, wherein the recorded nerve is a peripheral nerve.

50. A device network for modulating neural activity of a target nerve, comprising:
(a) one or more implantable devices in a first set of one or more implantable devices, comprising:
a sensor for detecting a detection signal, comprising an electrophysiological signal transmitted by a nerve or a physiological condition,
an ultrasonic transducer configured to actively transmit or backscatter ultrasonic waves, wherein the ultrasonic waves encode information related to the detection signal, and
a control circuit electrically coupled to the sensor and the ultrasonic transducer; and
(b) one or more implantable devices in a second set of one or more implantable devices, comprising:
a plurality of electrodes configured to emit an electrical pulse to a target nerve,
an ultrasonic transducer configured to receive ultrasonic waves encoding information related to the detection signal, and
a control circuit configured to extract the information related to the detection signal from the ultrasonic waves, and to operate the plurality of electrodes to emit the electrical pulse based on the information related to the detection signal;
wherein the one or more implantable devices in the first set of one or more implantable devices and the one or more implantable devices in the second set of one or more implantable devices are configured to wirelessly transmit information from the one or more implantable devices in the first set of one or more implantable devices to the one or more implantable devices in the second set of one or more implantable devices.

51. The device network of claim 50, wherein the one or more implantable devices in the first set of one or more implantable devices and the one or more implantable devices in the second set of one or more implantable devices are configured to wirelessly transmit information from the one or more implantable devices in the second set of one or more implantable devices to the one or more implantable devices in the first set of one or more implantable devices.

52. The device network of claim 50 or 51, wherein the device network comprises two or more implantable devices in the first set of one or more implantable devices.

53. The device network of any one of claims 50-52, wherein the device network comprises two or more implantable devices in the second set of one or more implantable devices.

54. The device network of any one of claims 50-53, wherein the control circuit of the one or more implantable devices in the first set of one or more implantable devices or the second set of one or more implantable devices is configured to determine whether to emit one or more electrical pulses from the one or more implantable devices based at least on information wirelessly received by the one or more implantable devices.

55. The device network of claim 54, wherein the control circuit of the one or more implantable devices in the first set of one or more implantable devices or the second set of one or more implantable devices is configured to select one or more pulse characteristics of the one or more electrical pulses.

56. The device network of claim 54 or 55, wherein determining whether to emit an electrical pulse comprises updating a dynamic state of the one or more implantable devices.

57. The device network of any one of claims 50-56, wherein the one or more implantable devices in the second set of one or more implantable devices further comprise a sensor for detecting an electrophysiological signal transmitted by a nerve or a physiological condition.

58. The device network of any one of claims 50-57, further comprising one or more intermediate devices comprising an ultrasonic transducer, wherein the one or more intermediate devices are configured to:
wirelessly receive the information from the one or more implantable devices in the first set of one or more implantable devices through ultrasonic waves, and
wirelessly transmit the information to the one or more implantable devices in the second set of one or more implantable devices through ultrasonic waves.

59. The device network of claim 58, wherein the one or more intermediate devices are further configured to:
wirelessly receive the information from the one or more implantable devices in the second set one or more implantable devices of implantable devices through ultrasonic waves, and
wirelessly transmit the information to the one or more implantable devices in the first set of one or more implantable devices through ultrasonic waves.

60. The device network of claim 58 or 59, wherein the one or more intermediate devices are configured to:
actively transmit ultrasonic waves to the one or more implantable device in the first set of one or more implantable devices;
receive backscattered ultrasonic waves that encode the information related to the detection signal detected by the sensor; and
actively transmit ultrasonic waves that encode the information related to the detection signal to the one or more implantable devices in the second set of one or more implantable devices.

61. The device network of any one of claims 58-60, wherein the intermediate device comprises a control circuit configured to:
extract the information related to the detection signal from the ultrasonic waves received by the intermediate device, and
determine whether one or more electrical pulses should be emitted from the one or more implantable devices in the second set of one or more implantable devices based at least on information wirelessly received by the one or more implantable devices in the first set of one or more implantable devices;
wherein the information related to the detection signal encoded in the ultrasonic waves transmitted from the intermediate device to the one or more implantable devices in the second set of one or more implantable devices comprises instructions to emit the one or more electrical pulses, and
wherein the control circuit of the one or more implantable devices in the second set of one or more implantable devices is configured to operate the plurality of electrodes to emit the electrical pulse based on the instructions.

62. The device network of claim 61, wherein the instructions to emit the one or more electrical pulses comprises instructions for one or more pulse characteristics of the one or more electrical pulses.

63. The device network of any one of claims 50-60, wherein:
the control circuit of the one or more implantable devices in the first set of one or more implantable devices is configured to determine whether one or more electrical pulses should be emitted from one or more implantable devices in the second set of one or more implantable devices based on at least the detection signal detected by the sensor of the one or more implantable devices in the first set of one or more implantable devices;
wherein the information related to the detection signal encoded in the ultrasonic waves actively transmitted or backscattered by the ultrasonic transducer of the one or more implantable devices in the first set of one or more implantable devices comprises instructions to emit the one or more electrical pulses; and
wherein the control circuit of the one or more implantable devices in the second set of one or more implantable devices is configured to operate the plurality of electrodes to emit the electrical pulse based on the instructions.

64. The device network of claim 63, wherein the instructions to emit the one or more electrical pulses comprises instructions for one or more pulse characteristics of the one or more electrical pulses.

65. The device network of any one of claims 50-60, wherein the control circuit of the one or more implantable devices in the second set of one or more implantable devices is configured to determine whether one or more electrical pulses should be emitted from the one or more implantable devices in the second set of one or more implantable devices based on at least the information related to the detection signal, and operate the plurality of electrodes to emit the electrical pulse based on the determination.

66. The device network of claim 65, wherein the control circuit is further configured to select one or more pulse characteristics of the one or more electrical pulses.

67. The device network of any one of claims 50-66, wherein the sensor comprises a plurality of electrodes configured to detect the electrophysiological signal.

68. The device network of any one of claims 48-67, wherein the sensor is configured to detect the physiological condition.

69. The device network of claim 68, wherein the physiological condition is a temperature, a respiratory rate, a strain, a pressure, a pH, a presence of an analyte, or an analyte concentration.

70. The device network of any one of claims 50-69, wherein the one or more implantable devices in the first set of one or more implantable devices comprises a first sensor configured to detect the physiological condition, and a second sensor comprising a plurality of electrodes configured to detect the electrophysiological signal.

71. The device network of any one of claims 50-70, wherein the ultrasonic transducer of the one or more implantable devices in the first set of one or more implantable devices or the ultrasonic transducer of the one or more implantable devices in the second set of one or more implantable devices is configured to receive ultrasonic waves that power the one or more implantable devices.
